(19) United States
(12) Patent Application Publication
(10) Pub. No.: US 2006/0110398 A1
(43) Pub. Date: May 25, 2006

(54) DAP-10 and uses thereof

(75) Inventors: Joseph H. Phillips JR., Palo Alto, CA (US); Nevila Hyka, Mountain View, CA (US)

Correspondence Address: DNAX RESEARCH, INC.;LEGAL DEPARTMENT, 901 CALIFORNIA AVENUE, PALO ALTO, CA, 94304, US

(73) Assignee: Schering Corporation, Kenilworth, NJ (US)

(21) Appl. No.: 11/284,685

(22) Filed: Nov. 22, 2005

Related U.S. Application Data
(60) Provisional application No. 60/630,707, filed on Nov. 24, 2004.

(51) Int. Cl. A61K 39/395; A61K 48/00

(52) U.S. Cl. 424/155.1; 514/44

(57) ABSTRACT

The present invention relates to methods for identifying and using modulators of DAP10 biological activity in vitro and in vivo that are useful in the treatment of cancer and autoimmunity.

This filing is a U.S. Patent Application which claims benefit of U.S. Provisional Patent Application No. 60/630,707, filed Nov. 24, 2004, which is incorporated herein by reference.

### TECHNICAL FIELD

The present invention relates to the fields of immunology and medicine. More particularly, the invention relates to the modulation of DAP10 activity to enhance antitumor activity and reduce autoimmunity in vivo, and identification of compounds that mediate such modulation.

### BACKGROUND OF THE INVENTION

The activation threshold of immune cells is regulated by activating and inhibitory signals received through recognition of self and foreign antigens. Genetic defects that affect activating or inhibitory receptors renders the immune system unable to distinguish between self and non self causing autoimmunity or abnormal response against infectious agents and transformed cells (1, 2).

Many activating receptors are multisubunit complexes in which the transmembrane adaptor proteins are responsible for transducing signals inside the cell. DAP10 is a transmembrane adaptor protein which associates with the activating receptor NKG2D in a multisubunit receptor complex expressed in hematopoietic cells (3). NKG2D-DAP10 receptor complex is expressed constitutively on NK, γδ T cells and NKT cells and innate stimuli can further upregulate its expression (3-6). Upon activation, CD8⁺ T cells and macrophages expresses NKG2D-DAP10 receptor which participates in regulation of adaptive immune response (7,8). The expression of NKG2D at the cell surface requires its association with DAP10 protein. This involves interaction between an acidic amino acid in the transmembrane region of DAP10 and a basic amino acid in the transmembrane domain of NKG2D protein (3). The expression patterns of NKG2D and DAP10 do not completely overlap, so it is possible that DAP10 associates with other yet unidentified receptors, especially in some myeloid cell populations. In humans, NKG2D associates exclusively with DAP10 (3,4), whereas mice express two different isoforms for NKG2D, a long form (NKG2D-1) which associates only with DAP10, and a short form (NKG2D-s) which has been shown to pair with both DAP10 and DAP12 (9,10).

Unlike DAP12 and other adaptor proteins which signal via the immunoreceptor tyrosine-based activation motif (ITAM), DAP10 has no ITAM, but it contains an YXXM motif involved in activation of PI3-kinase pathway (3). In NK cells, DAP10 signaling directly induces cytotoxicity and enhances cytokine production initiated via DAP12-associated receptors (11, 12). In T cells, it provides primarily costimulation for TCR-induced signals (5).

The identified ligands for NKG2D-DAP10 receptor complex are MHC class I like proteins including MICA, MICB and ULBPs in human and Rae-1, H60 and MULT-1 in rodents (4, 13, 14, 15). In general, they are minimally expressed in adult tissues and are upregulated in tumor cells and pathogen-infected cells. Ectopic expression of NKG2D ligands in tumor cells results in rejection of tumors from the host (16, 17). However, NKG2D ligands can be expressed in normal tissues as well, thus making unclear the exact role of the NKG2D-DAP10 complexes.

While DAP10 is expressed and characterized on a number of cell types known to be important players in immune responses, there is no definitive evidence of the physiological role played by this molecule.

### BRIEF SUMMARY OF THE INVENTION

The present invention relates to the modulation of DAP10, a cell surface signaling molecule on hematopoietic cells, to change the dynamics of an ongoing immune response or the initiation of a primary immune response. Typically, an immune response is humoral (antibody-mediated) or cellular (T cell-mediated). The humoral immune response is characterized by the activation of Th2 cells and production of IL-4 and IL-10, and ultimately leads to the activation of antigen-specific B cells and antibody production. A cellular immune response, on the other hand, is characterized by the activation of Th1 cells and the production of IFN-γ and IL-12, resulting in the proliferation and activation of cytotoxic T cells, usually CD8+ T cells. Each of these responses is designed to protect the host from certain pathogens and the regulation of these responses is critical to maintaining a healthy immune system. Unregulated or aberrant immune responses result in autoimmunity, various immunopathologies, as well as depressed or non-existent anti-tumor responsiveness. One identified regulator of immune responses is the T regulatory cell. T regulatory cells profoundly suppress host immune responses and induce self-tolerance. See, e.g., Roncarolo et al., Curr. Opin. Immunol. 12:676-683 (2000); Sakaguchi et al., Immunol. Rev. 182:18-32 (2001). T regulatory cells (“Tregs”) appear to have various phenotypes, and thus are consistently identifiable only by their functional activities. To date, no single molecule has been identified whose presence (or absence) definitively contributes to this regulation of T cell responses. The inventors disclose herein DAP10 as a molecule whose activity is critical to the regulation of cellular immune responses, and thus as a significant target for modulating of immune responses in vivo.

Thus, featured herein is a method for stimulating or augmenting a cell-mediated response to a tumor in a subject, comprising administering to a subject in need thereof an agent that inhibits or suppresses DAP10 biological activity. In one embodiment, the agent can inhibit or suppress the expression of DAP10. The agent can disrupt DAP10 intracellular signaling. In one embodiment, the agent disrupts the triggering of the PI3-kinase pathway by DAP10. The agent useful in this method can be a DAP10 transmembrane domain, a DAP10-specific antibody or biologically active fragment thereof, or a DAP10 siRNA. The tumor can be a primary or a metastatic tumor. In some embodiments, the tumor expresses NKG2D ligand or LAGE-1. In one embodiment, the agent enhances or augments the cytotoxicity of NK cells or NKT cells. The agent can stimulates increased proliferation of NK cells or NKT cells. In some embodiments, the agent stimulates increased cytokine production from NK cells or NKT cells of at least signature cytokine. Signature cytokine can include IL-4, IFN-γ, TNF-α, or IL-2.

Also featured herein is a method of increasing cellular cytotoxicity against a target, comprising administering to a cell an effective amount of an agent that modulates DAP10 activity, wherein the DAP10 activity is reduced or eliminated. The target can be a tumor cell. In some embodiments, the mediator of the cellular cytotoxicity is a NK cell or a NKT cell. The cell can be in a tissue or organism.

Further featured herein is a method for suppressing or inhibiting T regulatory cells, comprising administering to a subject in need thereof, an agent that inhibits DAP10 biological activity. In some embodiments, the agent can inhibit or suppress the expression of DAP10. The agent can disrupt DAP10 intracellular signaling. In one embodiment, the agent disrupts the triggering of the PI3-kinase pathway by DAP10. The agent useful in this method can be a DAP10 transmembrane domain, a DAP10-specific antibody or biologically active fragment thereof, or a DAP10 siRNA. In one embodiment, the subject has cancer.

Featured herein is a method of preventing or treating cancer, comprising administering to a subject in need thereof an effective amount of an agent that inhibits or suppresses DAP10 biological activity. In some embodiments, the cancer is skin cancer. In a particular embodiment, the cancer is chemically-induced.

Also featured herein is a method for identifying compounds that attenuate or ameliorate carcinogenesis, comprising: a) contacting a cell expressing DAP10 with a test compound; b) assess the cell for inhibition of DAP10 biological activity; and c) identify the test compound as a compound that attenuates or ameliorates carcinogenesis as one that downregulates DAP10 biological activity. In some embodiments, the carcinogenesis is skin carcinogenesis. In a particular embodiment, the carcinogenesis is chemically-induced carcinogenesis. In some embodiments, the biological activity of DAP10 is assessed by induction of PI3 kinase signaling, increased cytotoxicity of NK cells or NKT cells, increased proliferation of NK cells or NKT cells, or increased cytokine production by NK cells or NKT cells.

Further featured herein is a method for identifying compounds that inhibit tumor growth, comprising: a) contacting a cell expressing DAP10 with a test compound; b) assessing the cell for inhibition of DAP10 biological activity; and c) identifying the test compound as a compound that inhibit tumor growth as one that downregulates DAP10 biological activity. The tumor can be primary or metastatic tumor. In some embodiments, the tumor is a skin tumor. In one embodiment, the tumor is chemically-induced. The biological activity of DAP10 can be assessed by induction of PI3 kinase signaling, increased cytotoxicity of NK cells or NKT cells, increased proliferation of NK cells or NKT cells, or increased cytokine production by NK cells or NKT cells.

Featured herein is a method for identifying compounds that attenuate or ameliorate autoimmune disease, comprising a) inducing an experimental autoimmune disease in a DAP10 −/− mouse; b) administering a test compound to said DAP10−/−; c) assessing at least one autoimmune disease indication; and d) identifying a test compound as the compound that attenuates or ameliorates the autoimmune disease when the test compound reduces or eliminates the disease indication. In some embodiments, the experimental autoimmune disease is experimental allergic encephalomyelitis (EAE), collagen-induced arthritis, experimental autoimmune myocarditis, experimental autoimmune ovaritis, or experimental autoimmune testicularitis. Also featured herein is a method of preventing or treating an autoimmune disease, comprising administering an effective compound identified by this method.

### BRIEF DESCRIPTION OF THE DRAWINGS

FIG. 1 illustrates the targeting of DAP10 gene. (A). Schematic representation of DAP10 wt allele where exons 3 and 4 were replaced with a neoᵣ cassette of 1.4 kb in ES cells. By introducing a plasmid containing the cre recombinase gene the lox P flanked neoᵣ gene is removed. (B). Southern blot analysis. The ES clones were analyzed by Southern blot to confirm the excision of the neoᵣ gene before microinjection of blastocytes. The Southern screening utilizes both 5′ and 3′ probes which flank the DAP10 targeting vector. Genomic DNA was isolated from control, or mutant ES cells, digested with NcoI and hybridized with 5′ and 3′ probes. Wt allele (wt) is represented by a band of 6 kb, the targeted allele (HR) is the band of 4.0 kb and the CRE flipped allele (CF) is the band of 5.1 kb. (C). RT PCR analysis of DAP10KO lymphoid tissues. Total RNA was isolated from spleens, lymph nodes and thymuses of wt or DAP10KO mice and subjected to real time RT PCR analysis using specific primers for DAP10, NKG2D and DAP12 genes. mRNA levels were normalized to ubiquitin levels. (D). Expression of NKG2D receptor on NK1.1⁺ cells. Wt or DAP10KO splenocytes (upper panel) or NK cells activated with IL-2 for three days (lower panel) were stained with a cocktail of anti-NK1.1 FITC and anti-NKG2D PE mAbs and analyzed by flow cytometry.

FIG. 2 shows that DAP10-deficient mice are protected against chemically-induced cutaneous carcinoma. Four months after initiation of carcinogenesis, skin tumors were counted and their stage of development, papilloma and carcinoma, was determined based on clinical and histological criteria. Wt tumors were mix of carcinoma and papilloma whereas DAP10KO tumors were only papilloma. The picture shows the appearance of chemically-induced skin tumors in representative wt or DAP10KO mice. The differences were statistically significant, p>0.05 as determined by unpaired Student t-test.

FIG. 3 shows that NK1.1⁺ cells are the ultimate effector cells responsible for the efficient rejection of carcinoma tumors in DAP10KO mice. (A). Transplantation of wt carcinoma cells. Mice (n=10 per group) were injected s.c. with 5×10⁴ wt carcinoma cells and monitored for tumor growth every 2-3 days during a month. (B). Tumor growth in mice depleted in NK1.1⁺ cells. Anti-NK1.1 mAb (PK136) or isotype control (mouse IgG₁ kappa) were given i.v. every 5 days starting at day −2. Mice (n=5 per group) received 5×10⁴ wt carcinoma cells at day 0. (C). Tumor growth in mice depleted in NK cells. Anti-asialo GM1 polyclonal antibody or PBS were given i.v. at days −2, 2, 6, 10. Mice (n=5 per group) received 5×10⁴ wt carcinoma cells at day 0. P values (p>0.05) were determined by unpaired Student t-test. Data are shown as the mean of tumor volume±SEM, at a given time point.

FIG. 4 shows DAP10KO NK1.1⁺ cells kill efficiently carcinoma skin tumors. (A). Expression of NKG2D-ligands in DMBA-T carcinoma cells. Flow cytometry analysis of wt carcinoma cells stained with NKG2D-Ig fusion protein or an Ig control and a PE-conjugated secondary Ab. (B). In vitro killing of carcinoma skin tumors by NK1.1⁺ cells. NK, NKT or NK1.1⁺ cells were isolated from wt or DAP10KO splenocytes and cultured with IL-2 for 7 days. They were used as effectors in a cytotoxicity assay against DMBA-T cells at different effector:target ratios. The assay was performed in the presence of anti-NKG2D blocking mAb or the rat IgG2a isotype control, both used at 10 μg/ml.

FIG. 5 shows DAP10 deficiency is associated with enhanced immunity against B16 melanoma metastasis. (A). Development of pulmonary melanoma metastasis. 1×10⁵ or 1×10⁴ B16 cells were injected i.v. in mice and pulmonary metastasis were counted two weeks later, (p>0.05). (B). Appearance of B16 metastasis in the lungs of wt and DAP10KO mice at two weeks post-injection of 1×10⁵ of B16 melanoma cells.

FIG. 6 shows that NKT cells are the effectors responsible for protection against B16 melanoma metastasis in DAP10KO mice. (A). Generation of bone marrow chimeras. Four groups of bone marrow chimeras mice were generated and used to assess the role of DAP10KO immune cells in anti-metastatic effects. The donor→recipient chimeras used were the followings: 1. wt GFP⁺→wt; 2. DAP10KO→DAP10KO; 3. wt GFP⁺→DAP10KO; 4. DAP10KO→wt GFP⁺. 1×10⁵ B16 cells were injected in chimeras and two weeks later the metastasis were counted. Representative results from two different experiments. (B). Depletion of mice in NK1.1⁺ cells. Mice were injected i.v. with 2×10⁴ B16 cells. Anti-NK1.1 Ab (PK136) or isotype control (mouse IgG₁ kappa) were given i.v. every 5 days starting at day −2. The pulmonary metastasis were counted two weeks later. (C). Depletion of mice in NK cells. Anti-asialo GM1 polyclonal antibody or PBS were given i.v. at days −2, 2, 6, 10. 1×10⁵ B16 cells were injected i.v. at day 0 and pulmonary metastasis were counted at day 14.

FIG. 7 shows that DAP10KO NKT cells are hyperactive. (A). Flow cytometry analysis of NKT cell populations in wt and DAP10KO mice. Splenocytes isolated from naive mice were stained with anti-NK1.1 PE and anti-CD3 FITC antibodies. NKG2D expression was assessed in sorted NKT cells stained with anti-NKG2D PE Ab. (B). The turnover rate of NKT cells. Mice were injected i.v. with BrdU (1 mg per mouse). 24 hours later, leucocytes were isolated from spleens and bone marrow and stained to detect the BrdU incorporation using the BrdU kit. Stained cells were analyzed by flow cytometry. (C). DAP10KO NKT cells produce higher amounts of cytokines compared to wt cells. Wt or DAP10KO NKT cells were isolated from splenocytes of naive mice and activated in vitro with plate bound anti-CD3 mAb or isotype control (“resting”). Cells were cultured for 48 hours and cytokines production was measured in the supernatants using mouse Th1/Th2 CBA kit.

FIG. 8 shows DAP10 NKTKO cells efficiently kill B16 melanoma cells. (A). Flow cytometry analysis of activated NKTs. Sorted NKTs were cultured with IL-2 for 5 days. Cells were stained with a cocktail of anti-NK1.1 FITC, anti-CD3 Cy and anti-NKG2D PE Ab. (B). Analysis of NKTs cytotoxic capacities. IL-2-activated NKT cells were used as effector cells in a cytotoxicity assay against YAC-1 and B16 melanoma target cells. YAC-1 do express high levels of NKG2D ligands whereas B16 do not. The assay was performed in the presence of anti-NKG2D blocking Ab or the isotype control, both at 10 μg/ml.

FIG. 9 shows the depletion of CD4⁺CD25⁺ Tregs in wt and DAP10KO mice. (A). Transplantation of DMBA-T carcinoma cells in mice depleted in Tregs. To deplete in Tregs, mice received a single dose of anti-CD25 Ab at day −2 and then were injected with DMBA-T cells at day 0. Tumor growth was monitored every three days. (B). Development of metastasis in mice depleted in Tregs. For depletion, mice received anti-CD25 Ab, at days −3,2 and 7. Control mice received PBS. 1×10⁵B16 cells were given i.v. at day 0 and metastasis were count at two weeks. (C). The cytotoxic capacities of NKT cells isolated from mice depleted in Tregs. Wt or DAP10KO mice were depleted in CD25⁺ cells by intravenous injection of anti-CD25 Ab (clone PC61, 0.5 mg per mouse) at day −3. At day 0, NKT cells were isolated from spleens and cultured with mouse IL-2 for 5 days. Then, NKTs were used as effectors in a cytotoxicity assay against B16 melanoma cells. The data are shown as the means±SD.

FIG. 10 shows that the DAP10 deficiency is associated with impaired Treg-mediated suppression. (A). Flow cytometry analysis of Tregs. Splenocytes were isolated from wt or DAP10KO mice and stained with anti-CD4 FITC Ab, anti-CD25 biot Ab followed by Streptavidin-Cy. (B). Impaired cytokine production by DAP10KO Tregs. Wt or DAP10KO Tregs were isolated from splenocytes of naive mice and activated with IL-2 or IL-2 and soluble anti-CD3 mAb for 48 h. The production of cytokines was measured in the supernatants using the mouse inflammatory CBA kit. Other cytokines detected in the supernatants were MCP-1 (downregulated in DAP10KO Tregs) and TNF-α (similar production by wt and DAP10KO cells). (C). Taqman analysis of wt and DAP10KO Tregs. Tregs were isolated from spleens of wt and DAP10KO mice using mouse CD4⁺CD25⁺ Treg isolation kit. Total RNA was isolated from 3.5×10⁶ Tregs and subjected to real time RT PCR analysis using specific primers for Foxp3, NKG2D, DAP10 , DAP12 and IFNγ genes. mRNA levels were normalized to ubiquitin levels. (D). Adoptive transfer of wt Tregs in DAP10KO mice. 1×10⁶ wt Tregs or PBS was injected i.v. in DAP10KO mice at day −3. Mice were injected i.v. with B16 melanoma at day 0 and pulmonary metastasis analyzed at day 14. (D). The suppressive effect of Tregs on DAP10KO NKTs. NKTs were isolated from naive mice and cultured during 5 days with mouse IL-2. 12 hours prior the cytotoxicity assay, NKT cells were washed, counted and cultured either alone or with wt or DAP10KO Tregs (1:1 ratio) in media with IL-2. NKT±Tregs were used as effectors in a cytotoxicity assay against B16 melanoma cells or wt carcinoma cells. The data are shown as the means±SD.

FIG. 11 shows that DAP10KO T cells display increased reactivity to self peptide MOG. A. In vitro proliferation assay of MOG₃₅₋₅₅-immunized lymph node cells. Wt and DAP10KO lymph node cells were isolated from MOG₃₅₋₅₅-immunized mice and cultured with MOG₃₅₋₅₅ peptide at different concentrations during 72 hours. The proliferative response was measured by [³H]-thymidine incorporation assay, (left graph). Unlabeled cultures were used to determine the amount of IFN-γ (right graph). Data are means of triplicates±SEM. One representative experiment out of three is shown. B. Induction of DTH response. Mice were immunized with MOG emulsified in CFA at day 0 followed by foot pad injection of MOG on day 10. Food pad swelling was measured 48 h later (left graph). The EAE clinical score was determined as described in methods. Combined data of three distinct experiments.

### DETAILED DESCRIPTION OF THE INVENTION

The present invention relates to the physiological role for DAP10 as a critical regulatory of immune responses. The absence of DAP10 results in an augmentation of anti-tumor immune responsiveness both to primary outgrowth as well as metastatic tumor growth, implicating DAP10 as an important player in immunosurveillance. The absence of DAP10 also results in the heightened susceptibility to autoimmune disease, again implicating DAP10 as a participant in the regulation of immune responses in vivo. Thus, the object of the present invention relates to the modulation of DAP10 to alter immune responsiveness, particularly in cancer and autoimmunity. The ability to identify agents that modulate DAP10 permits the external regulation of immune response and may permit the enhancement of tumor immunosurveillance to at least reduce, if not prevent carcinogenesis and tumor growth.

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, published patent applications and other publications and sequences from GenBank and other databases referred to herein are incorporated by reference in their entirety. If a definition set forth in this section is contrary to or otherwise inconsistent with a definition set forth in patents, published patent applications and other publications and sequences from GenBank and other data bases that are herein incorporated by reference, the definition set forth in this section prevails over the definition that is incorporated herein by reference.

As used herein, “a” or “an” means “at least one” or “one or more.”

As used herein, the term “agent” includes compounds that modulate, e.g., up-modulate or stimulate and down-modulate or inhibit, the expression and/or activity of a molecule of the invention. As used herein the term “inhibitor” or “inhibitory agent” includes agents which inhibit the expression and/or activity of a molecule of the invention.

As used herein, the term “antibody” refers to an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab₂, so long as they exhibit the desired biological activity.

As used herein, the term “autoimmunity” refers to the condition in which a subject's immune system (e.g., T and B cells) starts reacting against his or her own tissues.

As used herein, the term “carcinogenesis” refers to the development of a malignant or neoplastic cell or tumor.

As used herein, the term “cell-mediated response” refers to a host response to an antigen, cell, or organism mediated by T cells as well as nonspecific cells of the immune system including but not limited to NK cells, macrophages, neutrophils, eosinophils, and basophils.

As used herein, the term “DAP10” encompasses all forms of DAP10 protein regardless of the source including but not limited to DAP10 as disclosed in the commonly owned WO99/06557.

As used herein, the term “metastatic tumor” refers to a tumor cell that grows at a site distant from the primary tumor.

As used herein, the term “NK cells” or “natural killer cells” refers to large granular lymphocytes that are distinct from B or T cells and function in innate immune responses to kill cells, typically either pathogen-infected or tumor, by various mechanisms including direct cell lysis, antibody dependent cell mediated lysis, and IFN-γ production to activate other cells.

As used herein, the term “NKT cell” refers to the small subset of T cells that also express NK cells markers. NKT cells are NK1.1 (CD161)+, CD3+, CD4+/− cells. The TCR-α chains have limited diversity relative to typical T cells, and the NKT cell recognizes class I-like molecules, such as CD1. NKT cells are not MHC restricted and do not recognize peptides displayed by antigen presenting cells.

As used herein, the term “peptide” includes relatively short chains of amino acids linked by peptide bonds. The term “peptidomimetic” includes compounds containing non-peptidic structural elements that are capable of mimicking or antagonizing peptides.

As used herein, the term “primary tumor” refers to a tumor that remains in situ.

As used herein, the term “regulatory T cell” or “Tregs” refers to T cells which cause suppression of other immune cells functions. See, e.g., von Herrath et al., Nature Rev. Immunol. 3:223-32 (2003). Tregs can suppress other immune functions either directly through cell-cell contact or indirectly through the secretion of anti-inflammatory mediators such as IL-10, TGF-β, or IL-4. See Levings et al., Arch. Allergy Immunol. 129: 23-76 (2002); Shevach, Nature Rev. Immunol. 2:389-400 (2002). In some embodiments, Tregs are CD4+CD25+ T cells. See, e.g., Waldmann et al., Immunity 14:399 (2001). Typically, Tregs actively suppress the proliferation and cytokine production of Th1, Th2, or naive T cells which have been stimulated in culture with an activating signal, e.g., antigen and antigen presenting cells or with a signal that mimics antigen in the context of MHC, e.g., anti-CD3 antibody, plus anti-CD28 antibody.

As used herein, the terms “small interfering RNA” (“siRNA”) or “short interfering RNAs”) refer to an RNA (or RNA analog) comprising between about 10-50 nucleotides (or nucleotide analogs) which is capable of directing or mediating RNA interference.

As used herein the term “subject” refers to any living organisms in which an immune response can be elicited, preferably the subjects are mammals. Exemplary subjects include, but are not limited to humans, monkeys, dogs, cats, mice, rats, cows, horses, goats and sheep.

As used herein, the term “T cell”, or “T lymphocyte” is refers to any cells within the T cell lineage from a mammal, e.g., human. Preferably, T cells are mature T cells that express either CD4 or CD8, but not both, and a T cell receptor. The various T cell populations described herein can be defined based on their cytokine profiles and their function as is known in the art.

As used herein, the term “treat” refers to the application or administration of a therapeutic agent to a subject, or application or administration of a therapeutic agent to an isolated tissue or cell line from a subject, who has a disease or disorder, a symptom of disease or disorder or a predisposition toward a disease or disorder, with the purpose of curing, healing, alleviating, relieving, altering, remedying, ameliorating, improving or affecting the disease or disorder.

As used herein, the term “tumor” refers to any malignant or neoplastic cell.

B. Modulation of Cellular Responses

Featured herein is a method for stimulating or augmenting a cell-mediated response to a tumor in a subject, comprising administering to a subject in need thereof an agent that inhibits or suppresses DAP10 biological activity. Also featured herein is a method of increasing cellular cytotoxicity against a target, comprising administering to a cell an effective amount of an agent that modulates DAP10 activity, wherein the DAP10 activity is reduced or eliminated.

Any cell-mediated response where DAP10 expressing cells participate can be stimulated or augmented using the disclosed methods. Such cell-mediated responses encompass naive, memory, Th1, Th2, and T regulatory cell responses. Cell-mediated responses can be measured by routine methods used in the art. See, e.g., Coligan et al., eds., CURRENT PROTOCOLS IN IMMUNOLOGY (John Wiley & Sons, current edition).

In particular, the manipulation of DAP10 activity can result in an increase of cellular cytotoxicity against a target. Any cytotoxic cell (or cytotoxic cell progenitor) that expresses DAP10, interacts with its target through a DAP10-mediated interaction, or whose differentiation and/or stimulation into a cytotoxic effector cell involves DAP10 can be modulated by this method. Such cells include but are not limited to NK cells, Tregs, NKT cells, CD4+ T cells, CD8+ T cells, macrophages, dendritic cells, and mast cells. Cellular cytotoxicity can be assessed by any suitable method. Exemplary methods include examining release of radiolabel from labeled target cells, e.g., ⁵¹Cr, colorimetric assays, e.g., CytoTox96® non-radioactive assay (Promega), granzyme release assays, lactate dehydrogenase assays, and bioluminescence cytotoxicity assays (e.g., Biovision Research Products (Mountain View, Calif.)).

Any suitable target cell can be a target for the cell-mediated response, particularly the cytotoxic, response of the present invention. Preferably, the target is mammalian. The target can be syngeneic, allogeneic, or xenogeneic to the responding cell. In most embodiments, the target is syngeneic or allogeneic. In a preferred embodiment, the target is syngeneic to the responding effector cell. The target cell can be a normal or abnormal cell. Exemplary cells include a tumor cell, a virally infected cell, and a cell expressing DAP10 ligand by recombinant means. Thus, target cells include but are not limited to established cell lines such as K562 cells, short term cell lines, or cells isolated from a sample taken from a subject, e.g., dissociated tumor cells. In cells which express DAP10 or a DAP10 ligand, the expression can be naturally occurring or by recombinant means using method known in the art. See, e.g., CURRENT PROTOCOLS IN MOLECULAR BIOLOGY (John Wiley & Sons, most recent edition).

In some embodiments, the tumor target or other cellular target expresses a ligand for DAP10. Sometimes, the DAP10 ligand (or DAP10 associated ligand) is NKG2D ligand or LAGE-1.

Further featured herein is a method for suppressing or inhibiting T regulatory cells, comprising administering to a subject in need thereof, an agent that inhibits DAP10 biological activity. Treg activity can be assessed using well known methods. See, e.g., U.S. Publication Nos. 2003/0049696; 2004/0173778; and 2003/0147865. Typically, effector T cell populations are assessed for proliferation, cytotoxicity, or cytokine production in response to a defined target in vitro. However, such analysis can also be performed ex vivo following in vivo administration of the agent. In such analyses, an effective amount of the agent is the amount necessary to increase proliferation and/or cytotoxicity of the desired effector cell population or alter the cytokine production to that which will support the effector cell population. In one example, the agent increases the proliferation and/or cytotoxicity of NK cells while decreasing IL-4 and IL-10 production and/or increasing IFN-γ production. In vivo analysis can include the augmentation of an anti-tumor response as assessed by reduced tumor growth, reduced incidence of metastasis, and/or reduced mortality. See, e.g., Teicher, TUMOR MODELS IN CANCER RESEARCH (Humana Press 2001).

The agent of the present invention can inhibit or suppress any aspect of DAP10 expression or function that results in an inhibition or suppression of DAP10 biological activity. The term “biological activity” refers to any immediate or downstream effect mediated by DAP10 interaction with a ligand. The effect can be a positive effect, e.g., initiates a signaling cascade when ligated, or a negative effect, e.g., the presence of a new or different signaling cascade that occurs when cell-cell interactions occur in the absence of DAP10 or the removal of a necessary signal for elicitation of a particular event. Thus, in some embodiments, the agent can disrupt DAP10 intracellular signaling. In one embodiment, the agent disrupts the triggering of the phosphoinositide 3-kinase (PI3-kinase) pathway by DAP10. PI3-kinase activity can be assessed by methods known in the art. Exemplary methods include assays examining protein phosphorylation, gene transcription, cell cycle progression, protein-protein interaction, e.g., ras, raf, or fyn, protein translocation, e.g., NF-κB translocation to the nucleus, or protein production assays, e.g., cytokine assays. See, e.g., Ausebel et al., eds., CURRENT PROTOCOLS IN MOLECULAR BIOLOGY (John Wiley & Sons, most recent edition). Functional assays such as proliferation and cytotoxicity can also be employed to demonstrate the final downstream effect of the agent using methods well known in the art and disclosed herewith. Proliferation can be assess using uptake of radiolabel, luminescence, fluorometry, and the like. Such a cell mediated response should be stimulated or augmented by at least about 5, 10, or 20%, sometimes about 30, 40, or 50%, and preferably greater than about 60%, 70%, 80%, 90%, 95%, or 99% following administration of the agent.

In one embodiment, the agent inhibits or suppresses the expression of DAP10. Such expression can occur through any mechanism including but not limited induction of a transcriptional repressor, inhibition of a transcriptional activator, and the like. DAP10 expression can be determined by any suitable means. Typically, DAP10 expression can be assessed by flow cytometric analysis. In some embodiments, complete suppression of expression may not be required to achieve a biologically relevant effect. Such an effect is one where the cell mediated response being targeted is detectably altered from the response that occurs in the absence of the administered agent. Such a response should be stimulated or augmented by at least about 5, 10, or 20%, sometimes about 30, 40, or 50%, and preferably greater than about 60%, 70%, 80%, 90%, 95%, or 99%.

In one embodiment, the agent enhances or augments the cytotoxicity of NK cells, NKT cells, or T cells. In some embodiments, the agent can stimulates increased proliferation of NK cells or NKT cells, and thereby stimulating or augmenting the cellular cytotoxic response.

The agent of the present invention can also stimulate or augment cell-mediated responses by stimulating increased cytokine production from one or more subsets of cells. In some embodiments, NK cells or NKT cells are stimulated to produce increased amounts at least signature cytokine relative to the cytokine expression seen in the absence of the agent. Signature cytokine can include IL-4, IFN-γ, TNF-α, or IL-2. These cytokines are readily detected in a quantitative fashion by any number of methods and kits commercially available including but not limited to ELISAs, microELISAs, and intracellular flow cytometric analysis. See, e.g., Coligan et al. (eds.), CURRENT PROTOCOLS IN IMMUNOLOGY (John Wiley & Sons, most recent edition).

The time, method, and vessel of contact can be any that are suitable to the function being assessed.

Exemplary agents that can inhibit or suppress DAP10 include but are limited to antibodies, RNAi, compounds that mediate RNAi, e.g., siRNA, antisense RNA, dominant/negative mutants of molecules of the invention, peptides such as the DAP10 transmembrane domain, and/or peptidomimetics.

In one embodiment, the agent of the present method is a DAP10 specific antibody or a biologically active fragment thereof. Exemplary antibodies include those disclosed in WO99/06557. The antibodies can be generated in cell culture, in phage, or in various animals, including but not limited to cows, rabbits, goats, mice, rats, hamsters, guinea pigs, sheep, dogs, cats, monkeys, chimpanzees, apes. Therefore, the antibody useful in the present methods is typically a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one V_{L} and one V_{H} region in the host cell. Exemplary descriptions of recombinant means of antibody generation and production include Delves, ANTIBODY PRODUCTION: ESSENTIAL TECHNIQUES (Wiley, 1997); Shephard, et al., MONOCLONAL ANTIBODIES (Oxford University Press, 2000); and Goding, MONOCLONAL ANTIBODIES: PRINCIPLES AND PRACTICE (Academic Press, 1993).

The antibody useful in the present methods can be modified by recombinant means to increase greater efficacy of the antibody in mediating a desired function such as increased half-life. See, e.g., Borrebaeck (ed.) ANTIBODY ENGINEERING (Oxford University Press, 1995). For example, antibodies can be modified by substitutions using recombinant means. Typically, the substitutions will be conservative substitutions. For example, at least one amino acid in the constant region of the antibody can be replaced with a different residue. See, e.g., U.S. Pat. No. 5,624,821, U.S. Pat. No. 6,194,551, Application No. WO99/58572; and Angal, et al., Mol. Immunol. 30: 105-08 (1993). The modification in amino acids includes deletions, additions, and substitutions of amino acids. The antibodies can also be fusion proteins where the antibody or biologically active fragment thereof is joined to another biologically relevant agent, e.g., a cytokine, an adhesion molecule, a costimulatory molecule, and the like as well as biologically relevant portions of such molecules.

RNA interference or RNAi″ refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes. See, e.g., U.S. Application No. 20040203145. Thus, RNAi directed to the expression of DAP10 itself, or any critical upstream or downstream effector for DAP10 expression or function are contemplated. In some embodiments, the RNAi can be used to modulate one or more components of the PI3-K signaling pathway used by DAP10.

Methods of making peptidomimetics based upon a known sequence are known in the art. See, e.g. U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Examples of unnatural amino acids which may be suitable amino acid mimics include but are not limited to β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-.alpha.-CBZ-L-lysine, N-ε-Boc-N-α-Fmoc-L-lysine, L-methionine sulfone, L-norleucine, L-norvaline, N-α-Boc-N-δ-CBZ-L-ornithine, N-δ-Boc-N-α-CBZ-L-ornithine, Boc-p-nitro-L-phenylalanine, Boc-hydroxyproline, and Boc-L-thioproline. Suitable peptidomimetics of a biologically relevant portion of DAP10, any protein critical to DAP10 biological activity including upstream and downstream signaling components, and the like.

In some embodiments, the agent useful in this method is a DAP10 transmembrane domain, a DAP10-specific antibody or biologically active fragment thereof, or a DAP10 siRNA. Also contemplated is the use of DAP10 fusion proteins comprising DAP10 or a biologically active fragment thereof and at least a protein domain or tag that facilitates transport, expression, biological activity, and the like for DAP10 in the host cell. Such proteins and tags can include but are not limited to Trans-Activating Transduction (TAT) proteins or other tags which promote protein entry into cells and transporter peptide tags. See, e.g., Vocero-Akabani et al., Methods Enzymol. 322:508-21 (2000).

In some embodiments, the subject has cancer and can be treated with the agent of the present invention as described below.

C. Methods of Identifying Agents That Modulate DAP Activity

Also featured herein is a method for identifying compounds that attenuate or ameliorate carcinogenesis, comprising: a) contacting a cell expressing DAP10 with a test compound; b) assess the cell for inhibition of DAP10 biological activity; and c) identify the test compound as a compound that attenuates or ameliorates carcinogenesis as one that downregulates DAP10 biological activity. In some embodiments, the carcinogenesis is skin carcinogenesis. In a particular embodiment, the carcinogenesis is chemically-induced carcinogenesis. In some embodiments, the biological activity of DAP10 is assessed by induction of PI3 kinase signaling, increased cytotoxicity of NK cells or NKT cells, increased proliferation of NK cells or NKT cells, or increased cytokine production by NK cells or NKT cells.

Further featured herein is a method for identifying compounds that inhibit tumor growth, comprising: a) contacting a cell expressing DAP10 with a test compound; b) assessing the cell for inhibition of DAP10 biological activity; and c) identifying the test compound as a compound that inhibit tumor growth as one that downregulates DAP10 biological activity. The tumor can be primary or metastatic tumor. In some embodiments, the tumor is a skin tumor. In one embodiment, the tumor is chemically-induced. The biological activity of DAP10 can be assessed by induction of PI3 kinase signaling, increased cytotoxicity of NK cells or NKT cells, increased proliferation of NK cells or NKT cells, or increased cytokine production by NK cells or NKT cells.

Any suitable DAP10 expressing cell may be used in the identifying methods of the present methods. DAP10 expression can be endogenous or exogenous. DAP10 expressing cells include, but are not limited to T cells, NK cells, NKT cells, dendritic cells, macrophages, and mast cells. For recombinant expression of DAP10, any suitable host cell may be employed. Host cells are genetically engineered (transduced or transformed or transfected) with DAP10 expression constructs using vectors with operably linked control regions suitable for the host cell. Exemplary sequences for DAP10 are disclosed in WO99/06557. Exogenous expression of DAP10 can be transient, stable, or some combination thereof. Exogenous expression can be enhanced or maximized by co-expression with one or more additional proteins, e.g., NKG2D. Exogenous expression can be achieved using constitutive promoters, e.g., SV40, CMV, and the like, as well as suitable inducible promoters known in the art. Suitable promoters are those which will function in the cell of interest. The vector may be, for example, in the form of a plasmid, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan. Representative host cells include, but are not limited to BaF cells, 293T cells, and murine mast cells.

Also provided herein are vectors or plasmids containing a nucleic acid that encodes for DAP10 or biologically active fragment thereof. Suitable vectors for use in eukaryotic and prokaryotic cells are known in the art and are commercially available or readily prepared by a skilled artisan. Additional vectors can also be found, for example, in CURRENT PROTOCOLS IN MOLECULAR BIOLOGY (Ausubel, et al., eds. 2000) and Sambrook et al., MOLECULAR CLONING: A LABORATORY MANUAL 2nd Ed. (1989).

Compounds that inhibit DAP10 biological activity are those molecules that reduce or eliminate at least one biological activity of DAP10. Such inhibition can occur through direct binding of one or more critical binding residues of DAP10 or through indirect interference including steric hindrance, enzymatic alteration of DAP10, and the like. As used herein, the term “compound” includes both protein and non-protein moieties. In one embodiment, the compound is a small molecule. In another embodiment, the compound is a protein.

A variety of different compounds may be identified using the method as provided herein. Compounds can encompass numerous chemical classes. In certain embodiments, they are organic molecules, preferably small organic compounds having a molecular weight of more than 50 and less than about 2,500 daltons. These compounds can comprise functional groups necessary for structural interaction with proteins, particularly hydrogen bonding, and may include at least an amine, carbonyl, hydroxyl or carboxyl group, preferably at least two of the functional chemical groups. The compounds can comprise cyclical carbon or heterocyclic structures and/or aromatic or polyaromatic structures substituted with one or more of the above functional groups. Compounds also include biomolecules like peptides, saccharides, fatty acids, steroids, purines, pyrimidines, derivatives, structural analogs or combinations thereof. Compounds of interest also can include peptide and protein agents, such as antibodies or binding fragments or mimetics thereof, e.g., Fv, F(ab′)₂ and Fab.

Compounds for the identification assay also can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

In one embodiment, small molecules can be used as compounds in the identification assay. Small molecule compounds include compounds that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides), and natural product extract libraries. In another embodiment, the compounds are small, organic non-peptidic compounds. In a further embodiment, a small molecule is not biosynthetic.

DAP10 expressing cells as provided herein are contacted using any convenient protocol with a compound (or agent). In some embodiments, the effects of the compound on DAP10-expressing cells are assessed in the presence of one or more types of other cells, e.g., labeled targets, proliferating T cells, and the like. In one embodiment, the cells are placed into a container that can hold a volume of a fluid medium, e.g., a well of a 96-well plate or 384 well plate, or an analogous structure. The cells and the compound can be contacted in any volume with any cell number that will permit accurate detection of DAP10 mediated events. In one embodiment, the total number of cells present ranges from about 1,000 to about 100,000 cells. In one embodiment, the reaction volume ranges from about 20 to 200 microliters. The cells can be contacted with the compound and/or other cell types for any period of time. In one embodiment, the time of contact ranges from one hour to eight hours, with a preferred time of four hours. The cells and the compound can be contacted in medium at any pH that is permissive for modulation of DAP10 biological activity. The cells can be contacted with the compound at various temperatures. In one embodiment, the temperature for contact of the cells often ranges from 25° C. to 38° C., with a temperature of 37° C. typically utilized. When desirable, the cells may be agitated to ensure adequate mixing and interaction of the various cell populations.

The amount of compound that is present in the contact mixture may vary, particularly depending on the nature of the compound. In one embodiment, where the agent is a small organic molecule, the amount of compound present in the reaction mixture can range from about 1 femtomolar to 10 millimolar. In another embodiment, where the agent is an antibody or binding fragment thereof, the amount of the compound can range from about 1 femtomolar to 10 millimolar. The amount of any particular compound to include in a given contact volume can be readily determined empirically using methods known to those of skill in the art.

The presence or absence of inhibition of DAP10 biological activity is determined by the assessment of DAP10 surface expression, DAP10 signaling, particularly through the PI3-K pathway, increased cytotoxicity against tumor or other abnormal cell targets, or increased cytokine production. The particular assessment protocol employed necessarily varies depending on the nature of the directly assessable assay and can employ any of the assays known in the art and disclosed herein in Section B. For example, where the assessed readout is DAP10 expression, DAP10 expressing cells that are either treated or untreated with a compound can be stained with DAP10 specific antibody, and the change in DAP10 expression assessed by standard flow cytometric analysis.

Any convenient means can be used to assess the effects of the compound on DAP10 biological activity including, but not limited to quantified measurements of proliferation, cytotoxicity, and expression in vitro either when contacted with compound alone or in the presence of other relevant cell types as well as assessment in vivo. Such assessment includes assessment in the ability to inhibit carcinogenesis and tumor growth and metastasis. Animal models suitable for such analysis are well known in the art and are exemplified by those disclosed in the Examples disclosed below. A compound is an inhibitor of DAP10 biological activity when the compound reduces the incidence of tumor development relative to that observed in the absence of the compound. In one embodiment, the compound reduces the incidence of tumor development after exposure to a carcinogen to 0%, conferring complete protection. Likewise, a compound is an inhibitor of DAP10 biological activity when the compound reduces the rate of tumor growth and/or the incidence of metastasis relative to the observed in the absence of the compound. In one embodiment, the rate of tumor growth is determined by measurement of tumor size. Typically, the tumor size is reduced by about 10%, 20%, 30%, 40%, 50%, 60%, 70%, 80%, 90%, or greater. The incidence of metastasis can be assessed by examining relative dissemination (e.g., number of organ systems involved) and relative tumor burden in these sites. Tumor metastasis can be reduced by about 10%, 20%, 30%, 40%, 50%, 60%, 70%, 80%, 90%, or greater. In some embodiments, the compound can be assessed relative to other compounds that do not impact DAP10 biological activity.

In one embodiment, a compound that mediates RNAi can be used to inhibit DAP10 biological activity. As discussed above, RNA interference is a post-transcriptional, targeted gene-silencing technique that uses double-stranded RNA (dsRNA) to degrade messenger RNA (mRNA) containing the same sequence as the dsRNA. The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available.

In another embodiment, the compound that mediates DAP10 inhibition or suppression is a DAP10 specific antibody or biologically active fragment thereof as described above. In yet another embodiment, the compound is a DAP10 transmembrane fragment as described above.

Featured herein is a method for identifying compounds that attenuate or ameliorate autoimmune disease, comprising a) inducing an experimental autoimmune disease in a DAP10−/− mouse; b) administering a test compound to said DAP10−/−; c) assessing at least one autoimmune disease indication; and d) identifying a test compound as the compound that attenuates or ameliorates the autoimmune disease when the test compound reduces or eliminates the disease indication.

As described below in the Example section, the DAP10−/− mouse displays a marked sensitivity to autoimmune disease when challenged with a self antigen. Other experimental autoimmune systems can also be employed using the DAP10−/− mouse. In some embodiments, the experimental autoimmune disease is experimental allergic encephalomyelitis (EAE), collagen-induced arthritis, experimental autoimmune myocarditis, experimental autoimmune ovaritis, or experimental autoimmune testicularitis. See, e.g., Lahita et al. (eds.), TEXTBOOK OF THE AUTOIMMUNE DISEASES (Lippincott, Williams, & Wilkins 2000) for a review of various experimental disease models at pages 753-841. Each of these animal models exemplifies at least one clinically relevant autoimmune disease, and therefore provides a suitable model for screening compounds that may alleviate or ameliorate one or more symptoms of an autoimmune disease.

For experimental allergic encephalomyelitis (EAE) is an animal model for human multiple sclerosis (MS). EAE is an experimentally induced disease that shares many of the same clinical and pathological symptoms of MS. See, e.g., Martin, et al., Ann. Rev. Immunol. 10:153-87 (1992). EAE can be induced in certain strains of mice by immunization with myelin in an adjuvant. The immunization activates CD4+ T cells specific for myelin basic protein (MBP) and proteolipid (PLP). Activated T cells then enter the central nervous system and causing a characteristic anatomic pathology as well as overt “clinical” signs, e.g., ascending hind limb paresis leading to paralysis, of the disease. The clinical signs of EAE can be evaluated based on a 0-5 scale of ascending severity of symptoms. See, e.g., Korngold, et al., Immunogenetics 24:309-15 (1986); Cua et al., Nature 421:744 (2003).

Therefore, a compound is an inhibitor of DAP10 biological activity when the compound reduces the incidence of at least one autoimmune symptom relative to that observed in the absence of the compound. In one embodiment, the compound reduces the incidence of at least one autoimmune symptom to 0%, conferring complete protection. Likewise, a compound is an inhibitor of DAP10 biological activity when the compound reduces the severity of autoimmune symptoms observed relative to the observed in the absence of the compound (or the in the presence of a non-DAP10 modulating compound). Typically, the severity of the symptoms is reduced by about 10%, 20%, 30%, 40%, 50%, 60%, 70%, 80%, 90%, or greater.

D. Methods of Treatment Using DAP10 Inhibitory Agents

Featured herein is a method of preventing or treating cancer, comprising administering to a subject in need thereof an effective amount of an agent that inhibits or suppresses DAP10 biological activity. In some embodiments, the cancer is skin cancer. In a particular embodiment, the cancer is chemically-induced.

The subject treated by the present methods includes a subject having an adenocarcinoma, leukemia, lymphoma, melanoma, sarcoma, or teratocarcinoma. The tumor can be a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Such tumors include, but are not limited to: neoplasma of the central nervous system: glioblastomamultiforme, astrocytoma, oligodendroglial tumors, ependymal and choroids plexus tumors, pineal tumors, neuronal tumors, medulloblastoma, schwannoma, meningioma, meningeal sarcoma: neoplasma of the eye: basal cell carcinoma, squamous cell carcinoma, melanoma, rhabdomyosarcoma, retinoblastoma; neoplasma of the endocrine glands: pituitary neoplasms, neoplasms of the thyroid, neoplasms of the adrenal cortex, neoplasms of the neuroendocrine system, neoplasms of the gastroenteropancreatic endocrine system, neoplasms of the gonads; neoplasms of the head and neck: head and neck cancer, oral cavity, pharynx, larynx, odontogenic tumors: neoplasms of the thorax: large cell lung carcinoma, small cell lung carcinoma, non-small cell lung carcinoma, neoplasms of the thorax, malignant mesothelioma, thymomas, primary germ cell tumors of the thorax; neoplasms of the alimentary canal: neoplasms of the esophagus, neoplasms of the stomach, neoplasms of the liver, neoplasms of the gallbladder, neoplasms of the exocrine pancreas, neoplasms of the small intestine, vermiform appendix and peritoneum, adenocarcinoma of the colon and rectum, neoplasms of the anus; neoplasms of the genitourinary tract: renal cell carcinoma, neoplasms of the renal pelvis and ureter, neoplasms of the bladder, neoplasms of the urethra, neoplasms of the prostate, neoplasms of the penis, neoplasms of the testis; neoplasms of the female reproductive organs: neoplasms of the vulva and vagina, neoplasms of the cervix, adenocarcinoma of the uterine corpus, ovarian cancer, gynecologic sarcomas; neoplasms of the breast; neoplasms of the skin: basal cell carcinoma, squamous carcinoma, dermatofibrosarcoma, Merkel cell tumor; malignant melanoma; neoplasms of the bone and soft tissue: osteogenic sarcoma, malignant fibrous histiocytoma, chrondrosarcoma, Ewing's sarcoma, primitive neuroectodermal tumor, angiosarcoma; neoplasms of the hematopoietic system: myelodysplastic syndromes, acute myeloid leukemia, chronic myeloid leukemia, acute lymphocytic leukemia, HTLV-1, and T-cell leukemia/lymphoma, chronic lymphocytic leukemia, hairy cell leukemia, Hodgkin's disease, non-Hodgkin's lymphomas, mast cell leukemia; neoplasms of children: acute lymphoblastic leukemia, acute myelocytic leukemias, neuroblastoma, bone tumors, rhabdomyosarcoma, lymphomas, renal and liver tumors.

Also featured herein is a method of preventing or treating an autoimmune disease, comprising administering an effective compound identified by this method. Non-limiting examples of autoimmune diseases and disorders having an autoimmune component that may be treated according to the invention include type 1 diabetes, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

Any subject can be treated with the methods and compositions provided herein. Such a subject is a mammal, preferably a human, in need of such treatment. Veterinary uses of the disclosed methods and compositions are also contemplated. Such uses would include prevention of carcinogenesis, treatment of cancer, and prevention and treatment of autoimmune diseases in domestic animals, livestock and thoroughbred horses.

Various pharmaceutical compositions and techniques for their preparation and use will be known to those of skill in the art in light of the present disclosure. For a detailed listing of suitable pharmacological compositions and associated administrative techniques one may refer to the detailed teachings herein, which may be further supplemented by texts such as REMINGTON: THE SCIENCE AND PRACTICE OF PHARMACY 20th Ed. (Lippincott, Williams & Wilkins 2003.

The formulation and delivery methods will generally be adapted according to the site and the disease to be treated. Exemplary formulations include, but are not limited to, those suitable for parenteral administration, e.g., intravenous, intraarterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. The dosage of the compounds of the invention will vary according to the extent and severity of the need for treatment, the activity of the administered composition, the general health of the subject, and other considerations well known to the skilled artisan.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds exhibiting high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition, age, weight, and therapeutic responsiveness. Dosage amount and interval may be adjusted individually to provide plasma levels of the active moiety sufficient to maintain the desired therapeutic effects, or minimal effective concentration (MEC). The MEC will vary for each compound but can be estimated from in vitro data; for example, the concentration necessary to achieve 50-90% inhibition of DAP10 biological activity using the assays described herein.

The mode of administration is not particularly important. In one embodiment, the mode of administration is an I.V. bolus. In another embodiment, the administration is topical. Alternately, one may administer the compound in a local rather than systemic manner, for example, via injection of the compound directly into a tumor or autoimmune lesion, often in a depot or sustained release formulation.

The modulatory agents of the invention can be administered alone or in combination with one or more additional agents. For example, in one embodiment, two or more agents described herein can be administered to a subject. In another embodiment, an agent described herein can be administered in combination with other immunomodulating agents. Examples of other immunomodulating reagents include antibodies that block a costimulatory signal, (e.g., against CD28, ICOS), antibodies that activate an inhibitory signal via CTLA4, and/or antibodies against other immune cell markers (e.g., against CD40, against CD40 ligand, or against cytokines), fusion proteins (e.g., CTLA4-Fc, PD-1-Fc), and immunosuppressive drugs, (e.g., rapamycin, cyclosporine A or FK506). In certain instances, it may be desirable to further administer other agents that upregulate immune responses, for example, agents which deliver T cell activation signals, in order elicit or augment an immune response. Such agents include, but are not limited to the co-administration of cytokines such as IL-2 and IFN-γ.

Agents as described herein can be incorporated into pharmaceutical compositions suitable for administration. Such compositions typically comprise the agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. See e.g., REMINGTON: THE SCIENCE AND PRACTICE OF PHARMACY 20th Ed. (Lippincott, Williams & Wilkins 2003). Except insofar as any conventional media or agent is incompatible with the active compound, use thereof in the compositions is contemplated. Supplementary active compounds can also be incorporated into the compositions.

### EXAMPLE

The expression pattern of NKG2D-DAP10 receptor complex and its ligands suggests that they might play distinct roles under physiological and pathological conditions. In this study, the generation of DAP10KO mice were used to investigate the importance of DAP10 in tumor immunity. It was unexpectedly observed that DAP10 deficiency enhanced anti-tumor immunity by affecting the regulatory functions of Tregs and anti-tumor effector functions of NK1.1⁺ cells.

Efficient immune surveillance against syngeneic tumors is often associated with increased reactivity to self due to impaired functioning of T regulatory (Treg) cells. The data included herein demonstrated that constitutive DAP10 costimulatory signaling was part of regulatory mechanisms that control immunity against tumors. Mice lacking DAP10 (DAP10KO) showed enhanced immune response against chemically-induced skin carcinoma and melanoma pulmonary metastasis, which resulted in delayed tumor growth and decreased tumor incidence, and protected them in the long term from tumor malignancies. DAP10KO natural killer T lymphocytes (NKT) and natural killer (NK) cells were the effectors responsible for this antitumor phenotype. DAP10 deficiency attenuated the suppressive capacities of Tregs and resulted in increased NKT functions, including cytokine production and cytotoxicity, leading to efficient killing of tumors. Adoptive transfer of wild type Tregs in DAP10KO mice restored tolerance to syngeneic tumors indicating that DAP10KO Tregs were dysfunctional in vivo. Furthermore, DAP10KO T cells showed increased autoreactivity, thereby rendering DAP10KO mice more susceptible to induced-autoimmune reactions. The data demonstrated that DAP10 constitutive signaling was important in adjusting the activation threshold of Tregs and NKTs to avoid autoreactivity, but also modulated anti-tumor mechanisms.

Material and Methods

Generation of DAP10 deficient mouse. The murine DAP10 genomic locus was identified by radiolabeled hybridization to a Lambda library made from 129/SV liver DNA. One Lambda clone was isolated which was then subcloned and sequenced for further characterization of the locus. Exons 3 and 4 are critical for signaling and expression of DAP10 protein at the cell surface. Exon 3 codes for the transmembrane region and exon 4 codes for the cytoplasmic domain of DAP10. A conventional replacement type targeting vector was designed to remove sequence for both exons while incorporating 5.4 kb of homologous sequence. Embryonic stem (ES) cells were electroporated with linearized DAP10 targeting vector. Appropriately transfected ES clones were used for injection after removal of the neomycin cassette. The ES clones were screened by Southern blot analysis. The probes used are the followings: 5′ flanking sense-DAP10 probe, 5′-CCAGAGACAGAGTCAAACTATGTAG-3′; 5′ flanking antisense-DAP10 probe, 5-CTGTGAGTTCAAGGCTAGCCTGG-3′; 3′ flanking sense-DAP10 probe, 5′-CTAGAGGAACCTTCTTCTGCC-3′; flanking antisense-DAP10 probe, 5′-GCTCTGGAGCCCTCCTGGT-3′. All experimental mice, DAP10KO mice and control C57BL/6J mice (Jaxon), were used at 6-10 weeks of age and matched by sex and age. Mice were housed in a pathogen-free animal facility. All animal procedures used in this study were approved by DNAX institutional animal care and use committee.

In vivo tumor models. As described previously (20), to induce skin tumorigenesis, wt and DAP10KO C57BL6 mice were treated with a single topical dose of 7,12 dimethyl benzanthracene (DMBA, Sigma), 50 μg of DMBA in 200 μl acetone was applied on the shaved skin of each mouse. One week after initiation of carcinogenesis, 30 μg of 12-O-tetradecanoyl-phorbolacetate (TPA, Sigma) in 200 μl acetone was applied on the skin of each mouse. The TPA was then applied twice weekly for 20 weeks. Tumors were allowed to progress to carcinoma stage and the viability of mice was analyzed during one year. Papilloma or carcinoma tumors were scored based on clinical criteria and histology characteristics. For transplantation experiment, chemically-induced skin tumor cells (DMBA-T) were adapted to tissue culture conditions and subsequently injected at various concentrations s.c. into mice. The dimensions of subcutaneous tumors were measured every 2-3 days using a caliper. The volume of the tumor was determined using the formula: length×width×width/2. Statistical differences between groups were evaluated by using the two tailed unpaired Student t-test when two groups were analyzed and two way ANOVA for more than two groups.

For induction of B16 pulmonary metastasis, B16 melanoma cells (ATCC) were harvested for injection when in the logarithmic growth phase (≦50% confluent). A single cell suspension was prepared using cell dissociation solution (Gibco) followed by removal of possible clumps through a disposable cell strainer. Wt and DAP10KO C57BL6 mice were injected i.v. with 1×10⁵ B16 cells or as indicated in the legends and then lungs were removed two weeks after tumor inoculation and metastasis were counted using a dissecting microscope. For in vivo depletion of NK1.1⁺ cells, mice were injected i.v. with anti-NK1.1 mAb clone PK136 (ATCC) at 0.5 mg per mouse on day −1 and then every three days. Mouse IgG1 kappa isotype control antibody was used to inject the control groups. To in vivo deplete Treg cells, anti-CD25 mAb clone PC-61 (ATCC) was injected i.v. at 0.5 mg per mouse at the indicated days (see figure legends). To in vivo deplete NK cells, mice were injected i.v. with 30 μl of anti-asialo GM1 Ab (Cederlane) at day −2 and then every 4 days. All antibodies used for in vivo studies were endotoxin free. Efficiency of in vivo depletions was verified by FACs analysis of splenic leucocytes.

Generation of bone marrow chimeras. Recipient mice were exposed to one dose of 1000 rad gamma irradiation. 1.5×10⁷ bone marrow cells isolated from donor mice were delivered i.v. into the tail vein of the recipient mice. At 6 and 8 weeks post-reconstitution, peripheral blood cells and spleen cells of recipient mice were analyzed by flow cytometry to assess the levels of reconstitution. Wt mice used in this experiment were GFP positive (kind gift of Dr. Brian Schaefer), facilitating the assessment of the reconstitution. At 8 weeks, 95-98% of recipient cells were derived from donors. Viability of bone marrow chimeras was 100%.

Taqman analysis. Lymph nodes, spleens and thymus of naive wt B1/6 and DAP10KO mice were homogenized in STAT 60 reagent (Te1-test Inc.) using Polytron. Total RNA was isolated by chloroform extraction followed by isopropanol precipitation. 5 μg total RNA was treated with DNAse mix: 5× first strand buffer (Gibco-BRL), RNAsin (Promega) and DNAse I, Rnase free (Boehringer). cDNAs were prepared by RT-PCR. For real time transcript quantification, the cDNA was use in a fluorogenic 5′ nuclease assay using the chemistry of TaqMan system on the GeneAmp® 5700 Sequence detector (Applied Biosystems). Gene specific Taqman® primers (F, R) were created using the Primer Express Software v.1.5. (Applied Biosystems) for the following: muDAP10-CCGGATGTGGGACTCTGTCT; TGGGCGCATACATACAAACAC; muDAP12-CCTGGTCTCCCGAGGTCAA; GGCGACTCAGTCTCAGCAATG,muNKG2D-TTCGTTGTTCGAGTCCTTGCT; ACTGGCTGAAACGTCTCTTTGAA, ubiquitin and 18 S rRNA as controls. Taqman® reaction included 200 nM of each primer, 1× SYBR GREEN PCR master mix (Applied Biosystem) and 10 ng cDNA. Primers for IFNγ and Foxp3 were designed using Primer Express (ABI), and selecting for sequences that have no cross-reactivity with other family members. The real time PCR consisted of one cycle of each 50° C. for 2 min and 95° C. for 10 min, followed by 50 cycles of 95° C. for 15 s and 60° C. for 1 min. Data analysis utilized the Sequence detector Software to determine threshold cycle and Cₜ.

In vitro functional analysis. NKT cells were sorted from splenocytes that were previously depleted in B cells using CD19 MACs beads (Miltenyi). The purity of sorted NKT cells was ≧95%. For the cytokine assay, NKT cells were activated with plate bound anti-CD3 Ab (10 μg/ml) or rat IgG2a isotype control in Iscoves media supplemented with 10% FCS, 100 mM Pen/Strep/Glu, 1 mM Sodium Pyruvate, 100 μM Non Essential Amino acids, 5.5×10⁻⁵ M 2-ME, and 25 mM Hepes. All culture reagents were from Gibco. Supernatants were collected 48 hours later and cytokines were measured.

NK cells were isolated from B cells-depleted splenocytes using anti-NK cell (DX5) MACS microbeads (Miltenyi). The purity of sorted cells used was >95%. For the cytotoxicity assay, NK and NKT cells were expanded in complete Iscoves medium containing 50 ng/ml mouse IL-2 (Peprotech). A standard 4 h ⁵¹Cr-release assay was performed at various effector to target ratio (E:T) as previously described (12).

CD4⁺CD25⁺ Tregs were isolated from splenocytes using the Treg isolation kit of Miltenyi (purity>96%). For the cytokine assay, Tregs were cultured at 2×10⁵ cells per well in the presence of IL-2 (20 ng/ml) or IL-2 and soluble anti-CD3 Ab (10 ug/ml) for 48 h. Supernatants were collected and cytokines were measured.

Flow cytometry and cytokine analysis. For flow cytometry phenotyping, mouse cells were incubated first with anti-CD16/CD32 Ab (clone 2.4G2) to block the Fc non-specific binding. Then, two or three color staining was performed using the following staining reagents: anti-CD3 FITC, anti-CD3 Cy (clone 17 A2), anti-CD4 FITC (clone GK1.5), anti-CD25 PE or anti-CD25 biotin Ab (clone PC61), anti-NK1.1 PE, anti-NK1.1 biotin, anti-NK1.1 FITC (clone PK136) and Streptavidin-cychrome (Cy). All the above reagents were from Pharmingen. Anti-NKG2D PE (clone CX5) was a generous gift of Dr. Lewis Lanier. The expression of NKG2D ligands in tumor cells was detected by staining cells with NKG2D fusion protein (R&D) followed by an anti-human secondary antibody. The BD mouse Th1/Th2 kit or inflammatory kit was used to measure the levels cytokines produced by NKT or Treg cells. Samples were analyzed in a FACS Calibur flow cytometer. CellQuest™ and BD CBA softwares were used to analyze the acquired data.

Induction of DTH response. To induce the DTH response, mice were injected s.c. with 50 μg mouse myelin oligodentrocyte glycoprotein (MOG)₃₅₋₅₅ peptide emulsified in CFA containing 1 mg of heat inactivated Mycobacterium. tuberculosis H37A (Difco Lab), at day 0. At day 10, mice were injected s.c. with 25 μg MOG₃₅₋₅₅ peptide into the dorsal left footpad and with PBS into the right one. The right and left footpad thickness was measured 48 hours later using a caliper-type engineer micrometer. Footpad swelling was calculated by subtracting the right footpad thickness from the left one. Animals were observed day by day and neurological effects were quantified on an arbitrary clinical score. EAE scoring was done as following: 0. nothing; 1. full limp tail; 2. hind limp weakness; 3. inability to right/single hind limb paresis (weakness); 4. inability to right/single hind limb paralysis; 5. bilateral hind limb paralysis; 6. moribund.

Proliferation assay. Mice were immunized s.c. with 50 μg of MOG₃₅₋₅₅ emulsified in CFA H37A. Lymph nodes (brachial, axillary, inguinal) were harvested nine days later and single cell suspensions were prepared. Lymph node cells were cultured in 96 well plates at 5×10⁵ cells/well with different concentrations of MOG₃₅₋₅₅ peptide for 72 h. The amount of IFN-γ in culture supernatants was measured by ELISA using anti-IFN-γ R46A2 mAb for capture and AN18-biot mAb for detection. To assess T cell proliferative responses, cultures were pulsed with 1 μCi/well [³H]-thymidine (Amersham) at 48 h and harvested 24 h later. Thymidine incorporation was measured in a β-scintillation counter.

Results

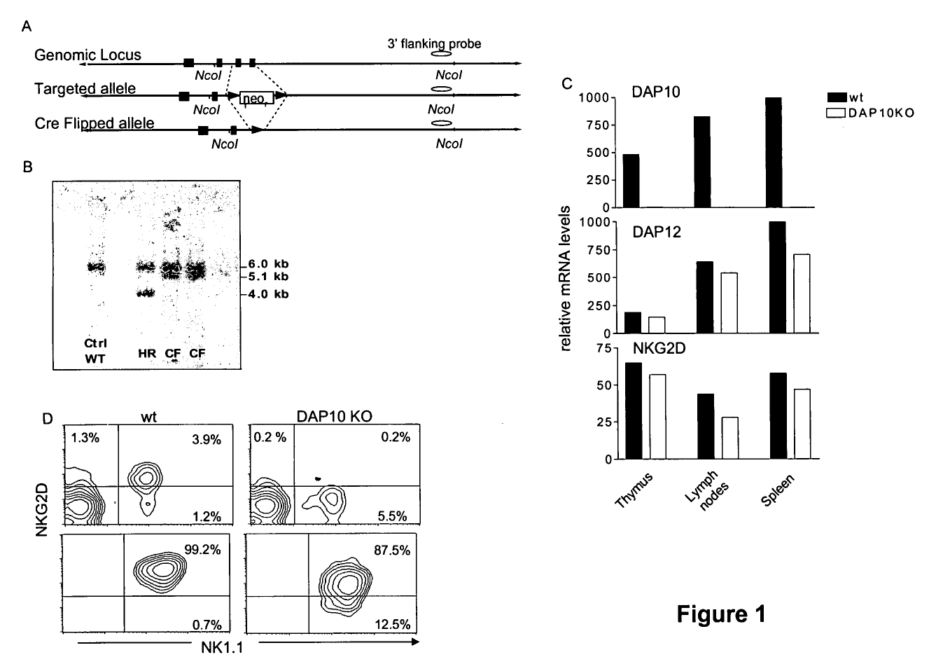

Generation of DAP10KO mice. The murine DAP10 gene maps to chromosome 7A3, adjacent and in opposite transcriptional orientation to the DAP12 gene. To generate DAP10-deficient mice, the DAP10 genomic locus was identified by radiolabeled hybridization to a lambda library made from 129/Sv liver DNA. One lambda clone was isolated which contained the loci for both DAP10 and DAP12. To disrupt the DAP10 gene, exons 3 and 4, which are critical for a functional DAP10 protein, were removed and replaced with a neomycin resistant gene (neoᵣ) in Bruce 4 C57BL/6J embryonic stem cells (FIG. 1A). The ES clones targeted appropriately were then transfected with a CRE recombinase expression vector to remove the neoᵣ cassette. Southern blot analysis confirmed the correct targeting of DAP10 gene (FIG. 1B). ES cells were injected into mouse blastocytes to generate the chimeric mice which were then intercrossed to obtain the homozygous animals. DAP10KO mice appeared to have in general normal organogenesis as assessed by histology. However, it was observed that DAP10KO spleens have increased weights compared to wt spleens and this difference was even more marked in mice immunized with adjuvant and self peptide (data not shown). This was a first sign suggesting that DAP10KO mice could be hyperimmune. DAP10 deficiency did not influence the basal expression of DAP12 or NKG2D genes at the mRNA level, as assessed by Taqman analysis of lymphoid tissues isolated from naive mice (FIG. 1C). Next, the expression of NKG2D protein at the cell surface, which normally requires its association with DAP10, was analyzed. FIG. 1D shows that constitutive expression of NKG2D protein is impaired in DAP10KO NK1.1⁺ splenocytes. DAP10KO NK1.1⁺ splenocytes, however, could express significant levels of NKG2D upon activation with IL-2 for 3 days (FIG. 1D). As recently reported (10), the short NKG2D isoform which pairs with DAP12 can be expressed as a functional surface receptor in activated DAP10KO splenocytes.

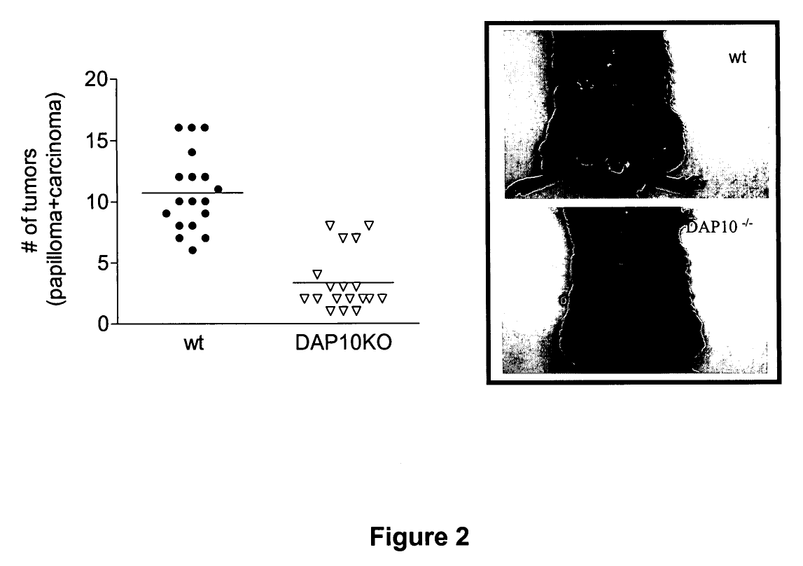

DAP10 deficiency confered protection to chemically-induced skin tumorigenesis and NK1.1⁺ cells were responsible for the antitumor effects. To study the role of DAP10 in immune surveillance during tumorigenesis, we used a chemically-induced skin tumor model. Mice were treated topically with a single dose of DMBA to initiate carcinogenesis, and then with TPA to promote proliferation of abnormal skin cells. The TPA treatment was performed during 4 months to allow progression of tumors from papillomas to carcinomas. As expected, wt mice developed large numbers of papillomas which grew in size and progressively became malignant carcinomas (FIG. 2). By contrast, DAP10KO mice developed significantly fewer papilloma type tumors that did not progress to the carcinoma stage (FIG. 2). One year after initiation of carcinogenesis, 65% of wt mice were dead from malignant skin carcinomas, whereas DAP10KO mice were healthy and protected from skin malignant transformation. Thus, unexpectedly, DAP10 deficiency conferred protection against chemically-induced skin malignancies.

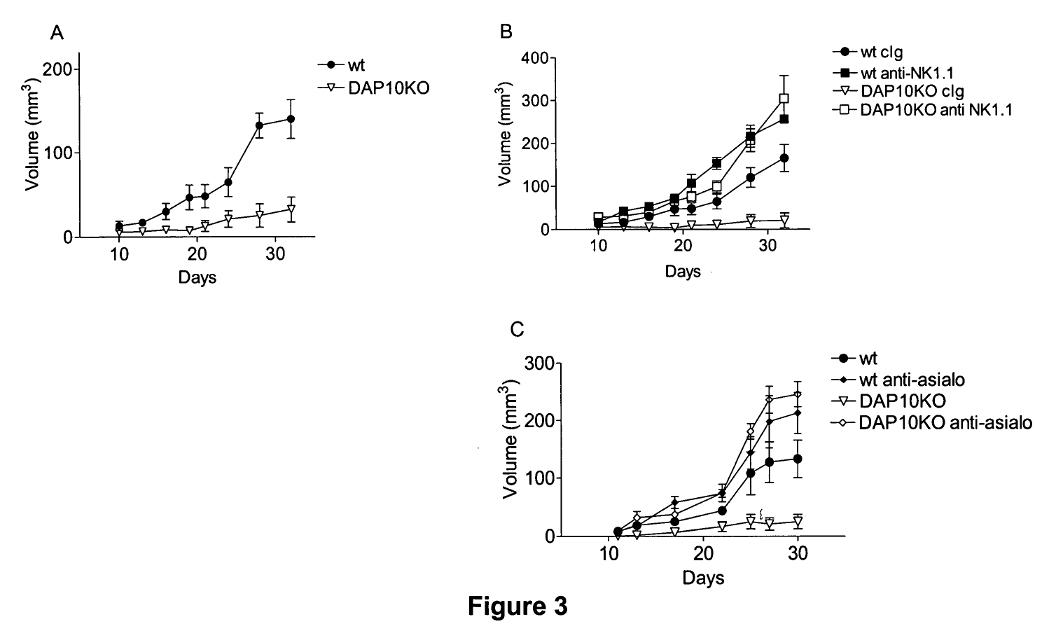

Because the chemically-induced skin tumor model is difficult to manipulate in long term in vivo depletion studies, a skin tumor transplantation model using a stable in vitro adapted DMBA-induced carcinoma cells line (DMBA-T) was employed to permit the analysis of the cellular mechanisms involved in DAP10KO antitumor response. Consistent with the tumorigenesis data, the subcutaneous inoculation of the wt carcinoma cells to DAP10KO mice resulted in markedly delayed tumor growth when compared to transplantation into wt mice (FIG. 3A). To determine if NK1.1⁺ cells were involved in the antitumor activity of DAP10KO mice, mice were treated with anti-NK1.1 Ab (PK136) to deplete them in NK1.1⁺ cells which includes both NK and NKT cells. Subcutaneous transplantation of DMBA-T cells to mice depleted of NK1.1⁺ cells resulted in similar tumor growth in wt and DAP10KO animals (FIG. 3B). These in vivo depletion results clearly suggested that NK1.1⁺ cells play a major role in DAP10KO antitumor effects. Since NK1.1 depletion removed both NK and NKT cells, tumor development in mice that were depleted only of NK cells by treatment with anti-asialo GM1 Ab were then analyzed. As shown in FIG. 3C, treatment of DAP10KO mice with anti-asialo GM1 Ab restored their susceptibility to carcinoma tumors. These findings indicated that NK cells were the primary effectors responsible for the delayed growth of the transplanted skin carcinoma in DAP10KO mice.

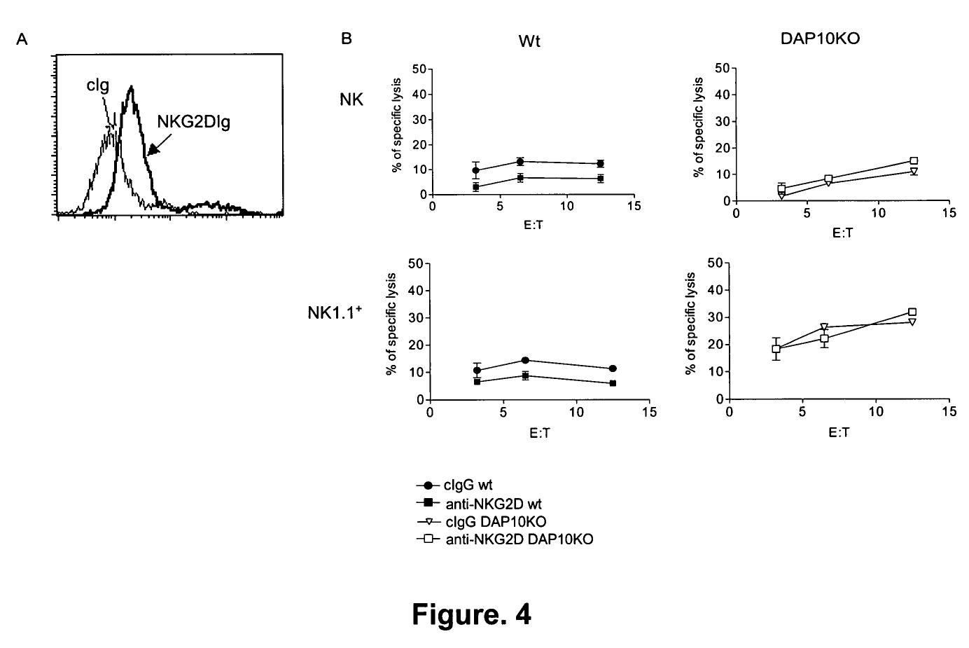

Although DMBA-T carcinoma cells expressed low levels of NKG2D ligands, they were unable to trigger an effective anti-tumor response in wt mice (FIG. 4A). To confirm the data obtained by the depletion experiments, the capacity of wt and DAP10KO NK and NK1.1⁺ cells to kill DMBA-T carcinoma cells in vitro was tested. Interestingly, activated DAP10KO NK1.1⁺ cells demonstrated substantially more cytolytic activity against DMBA-T carcinoma cells than wt cells, and this activity was mainly NKG2D-independent (FIG. 4B). Wt and DAP10KO NK cells appeared to kill DMBA-T carcinoma cells with similar efficiency, and their cytotoxicity was partially impaired by anti-NKG2D antibody (FIG. 4C). Although the in vivo experiments strongly suggested that NK cells in the DAP10KO are primarily responsible for anti-tumor activity, the in vitro generation of potent anti-DMBA-T cell cytotoxicity clearly involved NKT cells as well.

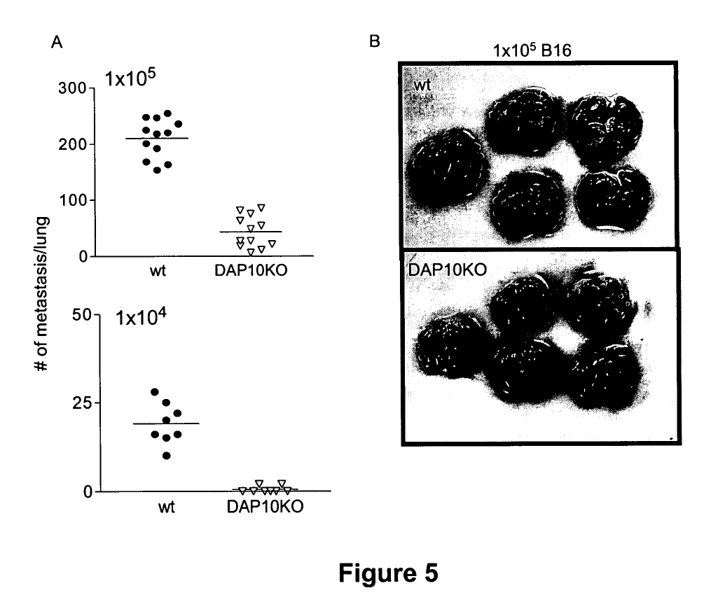

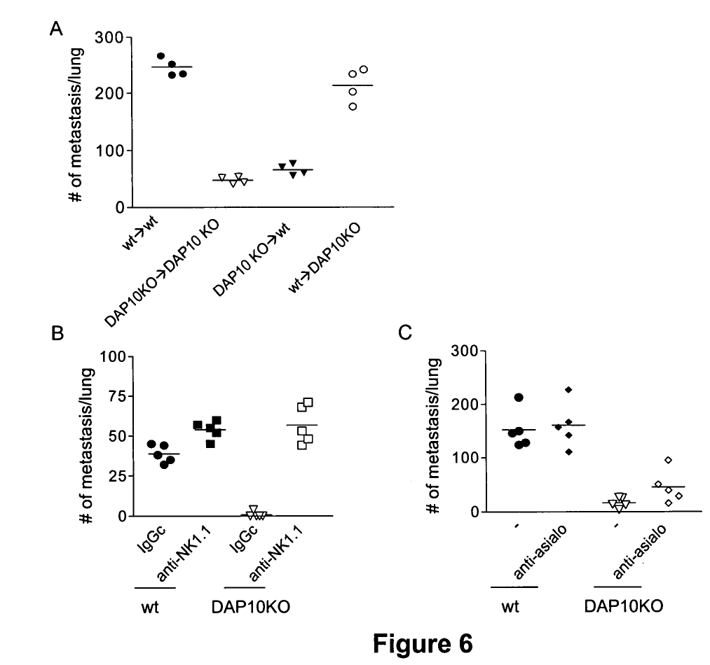

DAP10 deficiency conferred protection to skin metastasis and NKT cells were the ultimate effectors responsible for the antitumor phenotype. To further refine the antitumor activity of DAP10KO mice, the B16 melanoma metastasis model was employed. To induce pulmonary metastases, B16 cells were injected at various concentrations i.v. into wt and DAP10KO mice and two weeks later lungs were removed and analyzed for metastatic colonies. As shown in FIG. 5, DAP10KO mice injected with 1×10⁵ B16 cells developed 3-5 times fewer pulmonary metastases than wt animals. DAP10KO mice injected with 1×10⁴ B16 cells, were completely protected from melanoma metastasis. To investigate the hematopoietic origin of the antitumor activity in DAP10KO mice, bone marrow chimeras were established. DAP10KO mice reconstituted with wt bone marrow cells developed B16 metastasis at similar frequencies to those of wt animals (FIG. 6A). In contrast, reconstitution of wt mice with DAP10KO bone marrow cells resulted in resistance to metastasis comparable to that observed in DAP10KO animals (FIG. 6A). These results clearly demonstrated that hematopoietic cells were responsible for the DAP10KO antitumor activity.

In vivo depletion studies were performed to delineate the involvement of these cells in the DAP10KO enhanced antitumor activity against B16 metastasis. Depletion of NK1.1⁺ cells (NK and NKT cells) from wt animals resulted in a small but significant increase in lung B16 metastasis as compared to untreated wt animals (FIG. 6B). In addition, wt mice treated with anti-NK1.1 Ab presented B16 metastases in organs other than lungs, such as liver, skin, abdominal, and thoracic cavity, indicating that NK1.1⁺ cells in wt animals conferred some minor degree of protection from B16 melanoma metastasis. Interestingly, depletion of NK1.1⁺ cells from DAP10KO mice resulted in complete loss of antitumor activity with lung displaying B16 metastasis equivalent to wt animals.

To distinguish between NK cells and NKT cells functions in vivo, mice were treated with anti-asialo GM1 antibody to selectively deplete NK cells. In contrast to the NK1.1 depletions, removal of NK cells only from DAP10KO mice had essentially no effects on the antitumor activity of DAP10KO mice (FIG. 6C). These results suggest that NKT cells were the major effector population responsible for the defense against B16 melanoma metastases in DAP10KO mice.

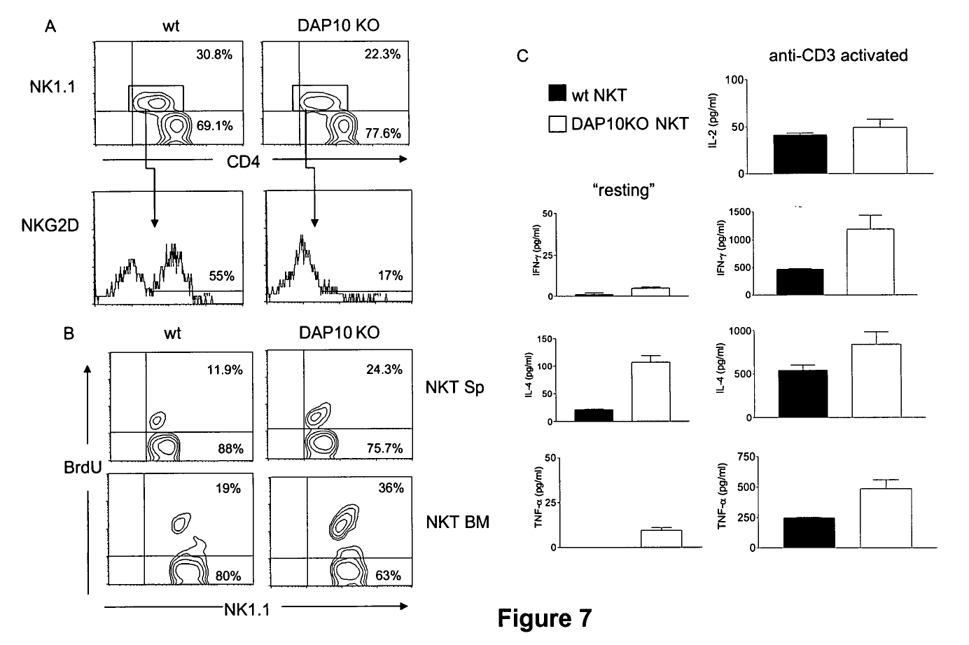

DAP10 deficiency caused NKT cell hyperactivity. Flow cytometry analysis revealed decreased frequencies of splenic DAP10KO NKT cells (FIG. 7A). It has been reported that in vivo activation of NKTs is associated with their rapid depletion from peripheral lymphoid organs followed by proliferation and repopulation from bone marrow-derived cells (23). Based on this knowledge, we analyzed the proliferative rate of splenic and bone marrow NKT cells in wt and DAP10KO mice by measuring the BrdU incorporation. As shown in FIG. 7B, DAP10KO NKT cells had a significantly increased proliferation percentages compared to wt cells, suggesting that they were constitutively hyperactive. To determine whether DAP10 deficiency affected the function of NKT cells, the capacity of wt and DAP10KO NKT cells to produce cytokines and kill tumor cells was tested. Because NKT cells were purified using anti-CD3 Ab which may slightly activate NKT cells, the function of resting NKT cells could not be truly assessed. As shown in FIG. 7C, NKT cells cultured with IgG isotype control antibody (“resting”), produced low levels of cytokines and thus were considered not activated. Stimulation of NKT cells with plate-bound anti-CD3 Ab induced the production of all NKT signature cytokines, including IL-4, IFNγ, TNF-α and IL-2 (FIG. 7C). Interestingly both nonactivated and activated DAP10KO NKT cells produced significantly higher amounts of cytokines when compared to wt cells.

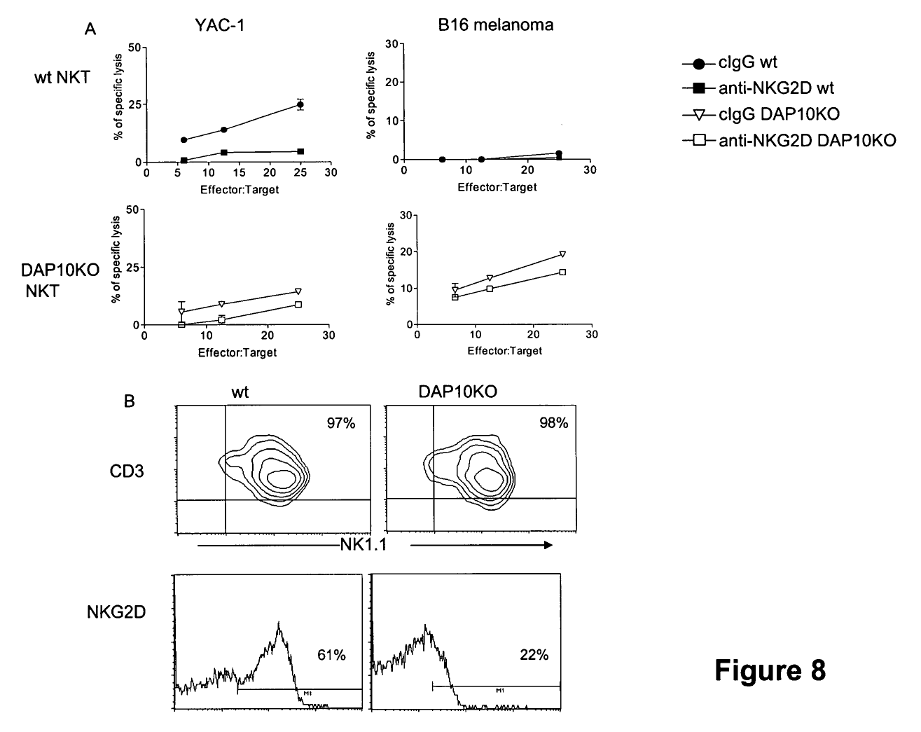

The cytotoxic capacity of IL-2-activated NKT cells to kill tumors expressing or not expressing NKG2D ligands were also tested. DAP10KO NKT cells killed less efficiently NKG2D ligand expressing YAC-1 tumor cells when compared to wt NKT cells, and anti-NKG2D Ab partially inhibited the killing in both cases (FIG. 8A). These results suggested that activated DAP10KO NKT cells expressed a partially functional NKG2D receptor, although the expression level for NKG2D was extremely low compared to wt cells (FIG. 8B). Interestingly, B16 melanoma cells were efficiently killed by DAP10KO NKT cells but not by wt cells, and this cytotoxicity was not affected significantly by anti-NKG2D Ab (FIG. 8A). These results were consistent with the in vivo data and indicated that DAP10KO NKT cells were programmed to efficiently kill these syngeneic tumor cells.

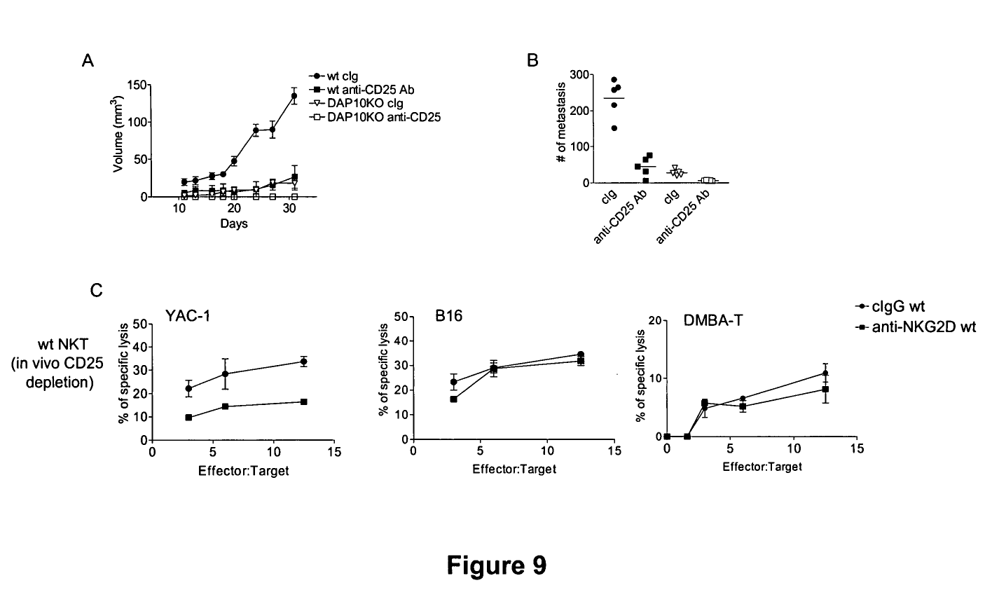

DAP10KO Tregs were dysfunctional. To understand the mechanistic pathways enabling DAP10-deficient mice to mount a better antitumor response, the effect of the DAP10 deficiency on the intrinsic properties of NK1.1⁺ cells, or the immune environment that controls their functions, was determined. The subcutaneous DMBA-T tumor growth in mice depleted in CD25⁺ Treg cells was analyzed. Wt mice depleted of Tregs with the in vivo anti-CD25 Ab treatment showed remarkably delayed and decreased skin carcinoma growth, likewise similar to what was observed in untreated DAP10KO mice (FIG. 9A, B). DAP10KO mice depleted in CD25⁺ Treg cells were completely free of tumors, demonstrating that DAP10KO Tregs have some suppressive activities. These enhanced anti-tumor activities were also observed in the B16 melanoma metastases model where depletion of Tregs blocked lung metastasis (FIG. 9B). Interestingly, wt NKT cells isolated from Treg-depleted mice were now capable of potent in vitro cytolysis of B16 melanoma cells and DMBA-T carcinoma cells comparable to the cytotoxicity mediated by DAP10KO NKT cells (FIG. 9C).

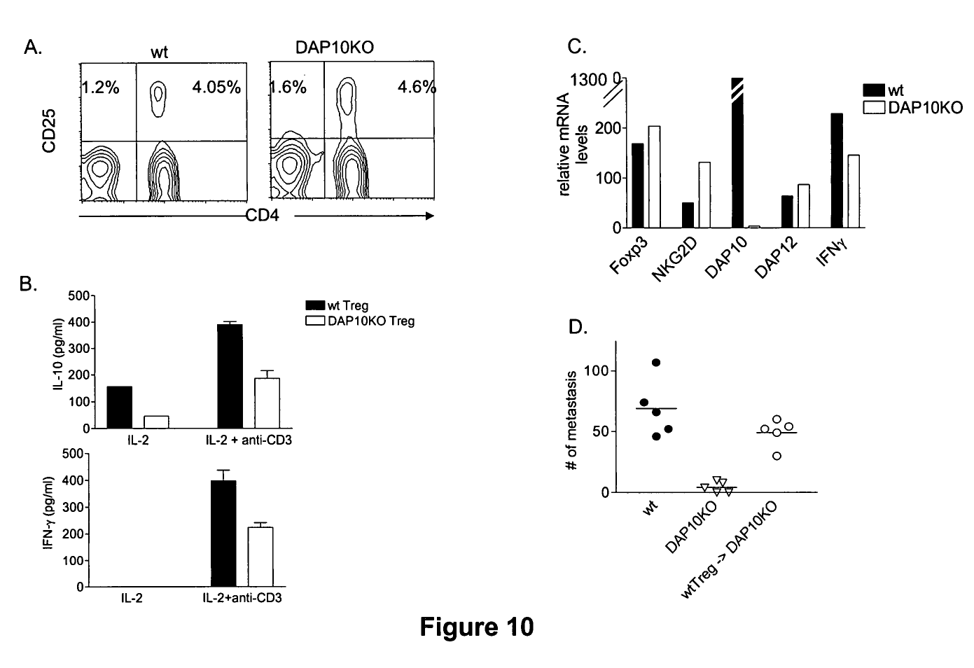

Phenotypic analysis of splenocytes from wt and DAP10KO mice showed equal percentages of CD4⁺CD25⁺ Tregs (FIG. 10A). DAP10KO Tregs, however displayed a higher proportion Tregs with low surface density CD25. Since CD25 is an important component of the IL-2 receptor, and IL-2 activation is critical for Treg differentiation and expansion, it was significant that DAP10KO Tregs activated with IL-2 or IL-2 plus soluble anti-CD3 Ab, produced lower amounts of IL-10 and IFN-γ when compared to wt cells (FIG. 10B). The expression of the NKG2D-DAP10 receptor complex in Tregs was then determined. As shown in FIG. 10C, we observed the presence of DAP10 and NKG2D transcripts in resting wt Tregs suggesting that DAP10 directly affected Treg functions. Surface expression of NKG2D receptor was undetectable in splenic T cells (not shown).

To determine whether DAP10KO Tregs were dysfunctional in vivo, wt Tregs were transferred into DAP10KO mice and analyzed their impact on the development of B16 melanoma metastasis. 1×10⁶ wt Treg cells were transferred i.v. into DAP10KO mice and 4 days latter mice received B16 melanoma tumor cells. Control mice, both wt and DAP10KO received only B16 cells. FIG. 10C shows that transfer of wt Tregs in DAP10KO mice rendered them susceptible to melanoma metastases comparable to wt mice.

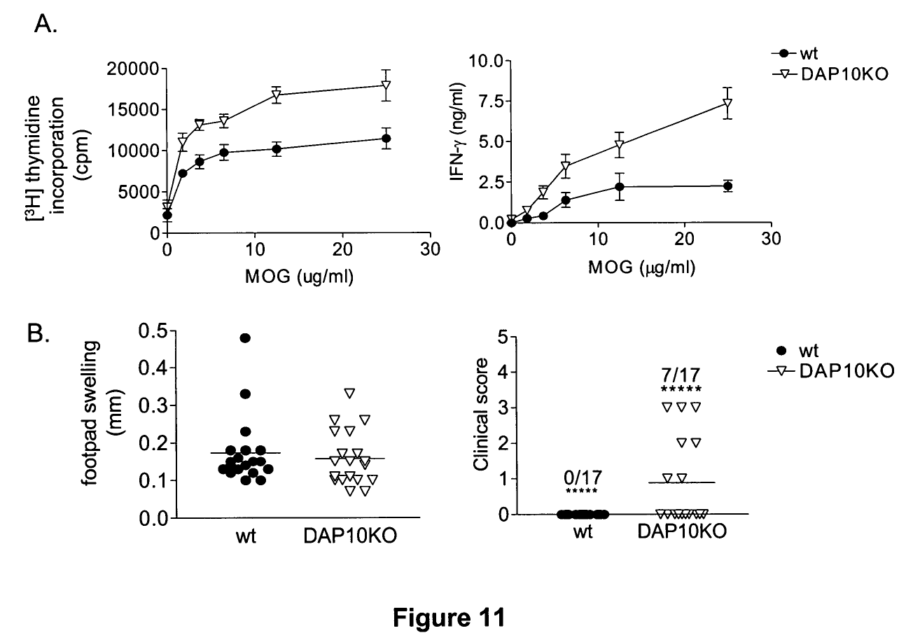

DAP10KO mice were more susceptible to induced-autoimmunity. To determine whether the impaired functioning of DAP10KO Tregs leads to increased reactivity to self, the in vitro and in vivo responses of DAP10KO T cells to a self antigen, MOG₃₅₋₅₅ were analyzed. FIG. 11A shows that, in a recall reaction to MOG₃₅₋₅₅ peptide, DAP10KO T cells isolated from lymph nodes of MOG₃₅₋₅₅-immunized mice, proliferated better and produced higher levels of IFNγ compared to wt cells. Induction of the delayed type hypersensitivity reaction (DTH) resulted in similar footpad swelling in wt and DAP10KO mice (FIG. 11B). Interestingly, the DTH response to MOG₃₅₋₅₅ triggered experimental autoimmune encephalomyelitis (EAE) in 40% of DAP10KO BL/6 mice but not in wt BL/6 mice. EAE-related neurological effects were observed as soon as 48 h after induction of the DTH response. Some DAP10KO mice developed mild EAE followed with rapid remission, whereas others had severe and persistent EAE that lasted one month before remission. Together the data suggested that DAP10KO mice had increased susceptibility to MOG₃₅₋₅₅-induced autoimmune response.

Discussion

Unexpectedly, DAP10KO mice exposed to carcinogenic stimuli or transplanted with syngeneic tumors expressing or not NKG2D ligands, showed enhanced resistance to tumor malignancies. The antitumor phenotype of DAP10KO mice was reversed by depleting mice in both NK and NKT cells. In the B16 melanoma model, DAP10KO NKT cells proved to be the effector cells mediating the elimination of B16 tumors. Consistently, in vitro cytotoxicity assays showed that DAP10KO, but not wt NKT cells, were able to efficiently kill B16 melanoma tumors in an NKG2D-independent way. By contrast, in the DMBA-T carcinoma transplantation model, DAP10KO NK cells were the ultimate effectors responsible for tumor rejection. However, in this tumor model as well, DAP10KO NKT cells appeared to mediate the activation of NK cells. Indeed, in vitro activated wt or DAP10KO NK cells killed DMBA-T tumors with similar efficiency, whereas DAP10KO NK1.1⁺ cells killed DMBA-T targets much more efficiently than wt NK1.1⁺ cells. In addition, upon stimulation through TCR, DAP10KO NKT cells produced higher levels of IFN-γ and TNF-α, which both have tumoricidal activities. In vivo, higher early production of IFN-γ by NKT cells was usually followed by rapid activation of NK cells and efficient tumor killing (31-35).

DAP10 deficiency was associated with constitutively hyperactive NKT cells. DAP10KO NKT cells showed an increased proliferation rate in both spleen and bone marrow of naive mice. In addition ‘resting’ as well as activated DAP10KO NKT cells produced significantly higher levels of cytokines compared to wt NKT cells. The functional properties of DAP10KO Tregs, known to prevent self destruction by inhibiting the activities of autoreactive T cells like NKT cells (36) were analyzed to determine if DAP10 participated in mechanisms that keep cytotoxic functions in check. The analysis of DAP10KO mice demonstrated that the basal expression of DAP10 was required for normal functioning of Tregs. A lack of DAP10 signaling appeared to alter the differentiation status of Tregs, resulting in a deficiency of their suppressive activities. The adoptive transfer of wt Tregs in DAP10-deficient mice restored the suppression, indicating that indeed DAP10KO Tregs were dysfunctional in vivo.

Tregs are highly differentiated cells expressing particularly high levels of CD25 antigen, a component of IL-2 receptor. IL-2 is not only a key growth factor for Tregs, but it can regulate their functions as well (27,29). Naive DAP10KO Treg population displayed an increased percentage of CD4⁺CD25^{low} cells when compared to wt cells. Consequently, IL-2-stimulation in conjunction or not to the activation through TCR, resulted in significant reduced production of IL-10 by DAP10KO Tregs. Tregs utilizes two main mechanisms to suppress autoreactivity: the production of the immunosuppressive cytokines like IL-10, TGFβ and IL-4 and the cell-cell contact interactions involving cell surface antigens as CTLA-4 or GITR (37). Thus, the impaired production of IL-10 by DAP10KO Tregs is likely to account for the decreased suppressive activities of those cells in vivo.

How does DAP10 signaling contribute in the differentiation process of Tregs? One possibility is that DAP10 affects the maturation of Tregs in the thymus. The thymic development of Tregs requires TCR-mediated signals and maximal costimulation. Mice lacking CD28, B7 and CD40 costimulatory molecules, have impaired development of Tregs (36,37). DAP10KO mice had normal Treg numbers, but impaired Treg suppressive functions, suggesting that DAP10 might be required for complete maturation of Tregs. DAP10 and NKG2D transcripts are well expressed in the thymus, and NKG2D-ligands transcripts are found to be highly expressed in the embryo (30), suggesting that NKG2D-DAP10 receptor complex can indeed be involved in early development of autoreactive T cell populations. Alternatively, DAP10 signaling either directly or indirectly may be required to maintain the suppressive functions of Tregs in the periphery. Although we were unable to detect the surface expression of NKG2D on natural wt Tregs, mRNA analysis clearly displayed the expression of both NKG2D and DAP10, suggesting a direct involvement of NKG2D-DAP10 receptor complex in Treg regulation. It is also possible that DAP10 signaling indirectly affects Treg functions by modulating the costimulatory receptors and cytokines provided by antigen presenting cells that are critical for the differentiation of Tregs in the periphery (32).

The analysis of DAP10KO mice suggest that DAP10 was involved in immune tolerance mechanisms of tumor recognition and thus represents a potential drug target for immunotherapy against tumors. Therefore, blocking DAP10 signaling might be protective against syngeneic tumors by impairing suppressive functions of Tregs. Of course, blocking NKG2D-DAP10 receptor complex is likely to have different outcomes depending on whether or not tumor cells express NKG2D ligands. Interestingly, wt mice were able to reject DMBA-T tumors which express low levels of NKG2D ligands, only when they were previously depleted in Tregs. It is possible that, besides NKG2D ligands, DMBA-T tumor cells express tumor associated antigens which can be recognized by Tregs and trigger their activation and subsequent inhibition of the immune reactions. This possibility is supported by the recent identification of a tumor specific, non mutated self antigen called LAGE-1 as the physiological ligand for Treg cells (38). Thus, the recognition of tumor specific antigens in a foreign or self context might have a critical impact on the nature of the immune response against tumors.

Because DAP10 has immunoregulatory functions, blocking DAP10 may favor autoimmunity. However, naive DAP10KO mice did not develop spontaneous autoimmune reactions, nor was the rejection of tumors in those mice associated with an autoimmune phenotype. Interestingly, induction of the DTH reaction to MOG self-peptide resulted in EAE in DAP10KO mice but not in wt mice, indicating that DAP10KO mice are more susceptible to experimentally induced-autoimmune diseases. It is well established that induction of EAE in BL/6 mice requires immunization with MOG₃₅₋₅₅ emulsified in CFA and Pertussis Toxin (PTX) (39). In the absence of PTX, wt BL/6 mice typically do not to develop EAE, however 40% of the DAP10KO mice developed EAE without PTX treatment. Presumably, impaired regulatory functions of DAP10KO Tregs resulted in enhanced T cell mediated-autoreactivity, therefore obviating the need for PTX. DAP10 signaling can also affect the activities of other immune cells involved in autoimmune response. Two studies have shown an expression of the NKG2D-DAP10 receptor complex in autoreactive CD8⁺ and CD4⁺ T cells (7,8). The latter made use of NOD mice which like other mice strains that are genetically susceptible to autoimmune diseases, have defective Tregs and NKT cells activities (28). Blocking NKG2D signaling prevented autoimmune diabetes by inhibiting the expansion and the activity of CD8⁺ T cells (8). Therefore, blocking DAP10 functions is likely to have different impacts on the immune response.

DAP10 is expressed broadly in lymphoid as well as myeloid cells and DAP10 biology is therefore expected to be complex. The study demonstrates an important role of DAP10 in the immunoregulatory mechanisms that control the response against syngeneic tumors and suggests that one physiological role of DAP10 costimulation is to activate Tregs to maintain tolerance to self antigens.

### REFERENCES

1. Walker L S, Abbas A K. 2002. The enemy within: keeping self-reactive T cells at bay in the periphery. Nat Rev Immunol. 2:11-19.
2. Lanier L L. Natural killer cell receptor signaling. 2003. Curr Opin Immunol. 15:308-314.
3. Wu J, Song Y, Bakker A B, Bauer S, Spies T, Lanier L L and Phillips J H. 1999. An activating immunoreceptor complex formed by NKG2D and DAP10. Science. 285(5428):730-732.
4. Bauer S, Groh V, Wu J, Steinle A, Phillips J H, Lanier L L and Spies T. 1999. Activation of NK cells and T cells by NKG2D, a receptor for stress-inducible MICA. Science. 285(5428):727-729.
5. Jamieson A M, Diefenbach A, McMahon C W, Xiong N, Carlyle J R and Raulet D H. 2002 The role of the NKG2D immunoreceptor in immune cell activation and natural killing. Immunity. 17(1):19-29.
6. Ho E L, Carayannopoulos L N, Poursine-Laurent J, Kinder J, Plougastel B, Smith H R and Yokoyama W M. 2002. Costimulation of multiple NK cell activation receptors by NKG2D. J Immunol. 169(7):3667-75.
7. Groh V, Bruhl A, El-Gabalawy H, Nelson J L, Spies T. 2003. Stimulation of T cell autoreactivity by anomalous expression of NKG2D and its MIC ligands in rheumatoid arthritis. Proc Natl Acad Sci. USA. 100(16):9452-7.
8. Ogasawara K, Hamerman J A, Ehrlich L R, Bour-Jordan H, Santamaria P, Bluestone J A, Lanier L L. 2004. NKG2D blockade prevents autoimmune diabetes in NOD mice. Immunity. 20(6):757-67.
9. Diefenbach A, Tomasello E, Lucas M, Jamieson A M, Hsia J K, Vivier E and Raulet D H. 2002 Selective associations with signaling proteins determine stimulatory versus costimulatory activity of NKG2D. Nat Immunol. 12:1142-1149.
10. Gilfillan S, Ho E L, Cella M, Yokoyama W M and Colonna M. 2002. NKG2D recruits two distinct adapters to trigger NK cell activation and costimulation. Nat Immunol. 12:1150-1155.
11. Billadeau D D, Upshaw J L, Schoon R A, Dick C J, Leibson P J. 2003. NKG2D-DAP10 triggers human NK cell-mediated killing via a Syk-independent regulatory pathway. Nat Immunol. 4:557-64.
12. Wu J, Cherwinski H, Spies T, Phillips J H and Lanier L L. 2000. DAP10 and DAP12 form distinct, but functionally cooperative, receptor complexes in natural killer cells. J Exp Med. 192(7):1059-68.
13. Cerwenka A, Bakker A B, McClanahan T, Wagner J, Wu J, Phillips J H and Lanier L L Retinoic acid early inducible genes define a ligand family for the activating NKG2D receptor in mice. Immunity. 12(6):721-727.
14. Cerwenka, A., O'Callaghan, C. A., Hamerman, J. A., Yadav, R., Ajayi, W., Roopenian, D. C., Joyce, S., Lanier, L. L. 2002. Cutting Edge: The Minor Histocompatibility Antigen H60 Peptide Interacts with Both H-2Kb and NKG2D. J Immunol. 168: 3131-3134.
15. Cosman, D., J. Mullberg, C. L. Sutherland, W. Chin, R. Armitage, W. Fanslow, M. Kubin, and N. J. Chalupny. 2001. ULBPs, novel MHC class I-related molecules, bind to CMV glycoprotein UL16 and stimulate NK cytotoxicity through the NKG2D receptor. Immunity. 14:123-133.
16. Cerwenka A, Baron J L and Lanier L L. 2001. Ectopic expression of retinoic acid early inducible-gene (RAE-1) permits natural killer cell-mediated rejection of a MHC class I-bearing tumor in vivo. Proc Natl Acad Sci USA. 98(20):11521-11526.
17. Diefenbach A, Jensen E R, Jamieson A M and Raulet D H. 2001. Rael and H60 ligands of the NKG2D receptor stimulate tumor immunity. Nature. 413(6852):165-171.
18. Malarkannan S, Horng T, Eden P, Gonzalez F, Shih P, Brouwenstijn N, Klinge H, Christianson G, Roopenian and D, Shastri N. 2000. Differences that matter: major cytotoxic T cell-stimulating minor histocompatibility antigens. Immunity. 13(3):333-344.
19. Hamerman J A, Ogasawara K, and Lanier L L. Cutting edge: toll-like receptor signaling in macrophages induces ligands for the NKG2D receptor. 2004. J Immunol. 15:172(4):2001-2005.
20. Oft M, Akhurst R J, Balmain A. 2002. Metastasis is driven by sequential elevation of H-ras and Smad2 levels. Nat Cell Biol. 4:487-94.
21. Smyth M J, Crowe N Y, Hayakawa Y, Takeda K, Yagita H, Godfrey D I. 2002. NKT cells—conductors of tumor immunity? Curr Opin Immunol. 14:165-71.
22. Cerwenka A, Lanier L L. 2001. Natural killer cells, viruses and cancer. Nat Rev Immunol. 1:41-49.
23. Eberl G, MacDonald H R. 1998. Rapid death and regeneration of NKT cells in anti-CD3epsilon- or IL-12-treated mice: a major role for bone marrow in NKT cell homeostasis. Immunity. 9(3):345-53.
24. Shimizu J, Yamazaki S, Sakaguchi S. 1999. Induction of tumor immunity by removing CD25+CD4+ T cells: a common basis between tumor immunity and autoimmunity. J Immunol. 163:5211-18.
25. Onizuka S, Tawara I, Shimizu J, Sakaguchi S, Fujita T, Nakayama E. 1999. Tumor rejection by in vitro administration of anti-CD25 (Interleukin-2 receptor a) monoclonal antibody. Cancer Res. 59:3128-33.
26. Azuma T, Takahashi T, Kunisato A, Kitamura T, Hirai H. 2003. Human CD4⁺CD25⁺ regulatory T cells suppress NKT cell functions. Cancer Res. 63:4516-20.
27. Furtado G C, Curotto de Lafaille M A, Kutchukhidze N, Lafaille J J. 2002. Interleukin 2 signaling is required for CD4+ regulatory T cell function. J. Exp. Med. 196:851-57.
28. Bluestone J A, Abbas A K. 2003. Natural versus adaptive regulatory T cells. Nat Rev Immunol. 3:253-7.
29. Malek T R. 2003. The main function of IL-2 is to promote the development of T regulatory cells. J Leukoc Biol. 74:961-5.
30. Cerwenka A, Lanier L L. 2003. NKG2D ligands: unconventional MHC class I-like molecules exploited by viruses and cancer. Tissue Antigens. 61:335-43.
31. Smyth M J, Takeda K, Hayakawa Y, Peschon J J, van den Brink M R, Yagita H. 2003. Nature's TRAIL—on a path to cancer immunotherapy. Immunity. January; 18:1-6.
32. Walker L S. 2004. CD4⁺CD25⁺ Treg: divide and rule? Immunology. 111(2):129-37.
33. Smyth M J, Crowe N Y, Pellicci D G, Kyparissoudis K, Kelly J M, Takeda K, Yagita H, Godfrey D I. 2002. Sequential production of interferon-gamma by NK1.1⁺ T cells and natural killer cells is essential for the antimetastatic effect of alpha-galactosylceramide. Blood. February 15;99(4):1259-66.
34. Smyth M J, Thia K Y, Street S E, Cretney E, Trapani J A, Taniguchi M, Kawano T, Pelikan S B, Crowe N Y, Godfrey D I. 2000. Differential tumor surveillance by natural killer (NK) and NKT cells. J Exp Med. 191:661-8
35. Blankenstein T, Qin Z The role of IFN-gamma in tumor transplantation immunity and inhibition of chemical carcinogenesis. 2003. Curr Opin Immunol. 15:148-54.
36. Lohr J, Knoechel B, Jiang S, Sharpe A H, Abbas A K. 2003. The inhibitory function of B7 costimulators in T cell responses to foreign and self-antigens. Nat Immunol. 4:664-9.
37. Sakaguchi S. 2004. Naturally arising CD4⁺ regulatory T cells for immunologic self-tolerance and negative control of immune responses. Annu Rev Immunol. 22:531-62.
38. Wang H Y, Lee D A, Peng G, Guo Z, Li Y, Kiniwa Y, Shevach E M, and Wang R F. 2004. Tumor-specific human CD4+ regulatory T cells and their ligands: implications for immunotherapy. Immunity. 20:107-18.
39. Harald H. Hofstetter, Diane L. Sewell, Frances Liu, Matyas Sandor, Thomas Forsthuber, Paul V. Lehmann and Zsuzsa Fabry. 2003. Autoreactive T cells promote post-traumatic healing in the central nervous system. Journal of Neuroimmunol. 135: 25-34

What is claimed is:

1. A method for stimulating or augmenting a cell-mediated response to a tumor in a subject, comprising administering to a subject in need thereof an agent that inhibits or suppresses DAP10 biological activity.

2. The method of claim 1, wherein the agent inhibits or suppress the expression of DAP10.

3. The method of claim 1, wherein the agent disrupts DAP10 intracellular signaling.

4. The method of claim 3, wherein the agent disrupts the triggering of the PI3-kinase pathway by DAP10.

5. The method of claim 1, wherein the agent is a DAP10 transmembrane domain, a DAP10-specific antibody or biologically active fragment thereof, or a DAP10 siRNA.

6. The method of claim 1, wherein the tumor is a primary or a metastatic tumor.

7. The method of claim 1, wherein the tumor expresses NKG2D ligand.

8. The method of claim 1, wherein the tumor expresses LAGE-1.

9. The method of claim 1, wherein the agent enhances or augments the cytotoxicity of NK cells or NKT cells

10. The method of claim 1, wherein the agent stimulates increased proliferation of NK cells or NKT cells.

11. The method of claim 1, wherein the agent stimulates increased cytokine production from NK cells or NKT cells of at least signature cytokine.

12. The method of claim 11, wherein the signature cytokine is IL-4, IFN-γ, TNF-α, or IL-2.

13. A method of increasing cellular cytotoxicity against a target, comprising administering to a cell an effective amount of an agent that modulates DAP10 activity, wherein the DAP10 activity is reduced or eliminated.

14. The method of claim 13, wherein the target is a tumor cell.

15. The method of claim 13, wherein the mediator of the cellular cytotoxicity is a NK cell or a NKT cell.

16. The method of claim 13, wherein the cell is in a tissue or organism.

17. A method for suppressing or inhibiting T regulatory cells, comprising administering to a subject in need thereof, an agent that inhibits DAP10 biological activity.

18. The method of claim 17, wherein the agent inhibits or suppress the expression of DAP10.

19. The method of claim 17, wherein the agent disrupts DAP10 intracellular signaling.

20. The method of claim 19, wherein the agent disrupts the triggering of the PI3-kinase pathway by DAP10.

21. The method of claim 17, wherein the agent is a DAP10 transmembrane domain, a DAP10-specific antibody or biologically active fragment thereof, or a DAP10 siRNA.

22. The method of claim 17, wherein the subject has cancer.

23. A method of preventing or treating cancer, comprising administering to a subject in need thereof an effective amount of an agent that inhibits or suppresses DAP10 biological activity.

24. The method of claim 23, wherein the cancer is skin cancer.

25. The method of claim 23, wherein the cancer is chemically-induced.

26. A method for identifying compounds that attenuate or ameliorate carcinogenesis, comprising:
a) contacting a cell expressing DAP10 with a test compound;
b) assess the cell for inhibition of DAP10 biological activity; and
c) identify the test compound as a compound that attenuates or ameliorates carcinogenesis as one that downregulates DAP10 biological activity.

27. The method of claim 26, wherein the carcinogenesis is skin carcinogenesis.

28. The method of claim 26, wherein the carcinogenesis is chemically-induced carcinogenesis.

29. The method of claim 26, wherein the biological activity of DAP10 is assessed by induction of PI3 kinase signaling, increased cytotoxicity of NK cells or NKT cells, increased proliferation of NK cells or NKT cells, or increased cytokine production by NK cells or NKT cells.

30. A method for identifying compounds that inhibit tumor growth, comprising:
a) contacting a cell expressing DAP10 with a test compound;
b) assess the cell for inhibition of DAP10 biological activity; and
c) identify the test compound as a compound that inhibit tumor growth as one that downregulates DAP10 biological activity.

31. The method of claim 30, wherein the tumor is primary or metastatic tumor.

32. The method of claim 30, wherein the tumor is a skin tumor.

33. The method of claim 30, wherein the tumor is chemically-induced.

34. The method of claim 30, wherein the biological activity of DAP10 is assessed by induction of PI3 kinase signaling, increased cytotoxicity of NK cells or NKT cells, increased proliferation of NK cells or NKT cells, or increased cytokine production by NK cells or NKT cells.

35. A method for identifying compounds that attenuate or ameliorate autoimmune disease, comprising
a) inducing an experimental autoimmune disease in a DAP10−/− mouse;
b) administering a test compound to said DAP10−/−;
c) assessing at least one autoimmune disease indication; and
d) identifying a test compound as the compound that attenuates or ameliorates the autoimmune disease when the test compound reduces or eliminates the disease indication.

36. The method of claim 35, wherein the experimental autoimmune disease is experimental allergic encephalomyelitis (EAE), collagen-induced arthritis, experimental autoimmune myocarditis, experimental autoimmune ovaritis, or experimental autoimmune testicularitis.

37. A method of preventing or treating an autoimmune disease, comprising administering an effective compound identified by the method of claim 35.

38. A method of preventing or treating an autoimmune disease comprising administering to a subject in need thereof an effective amount of a compound that maintains, enhances, or restores DAP10 biological activity in DAP10 expressing cells.

39. A method of identifying compounds to prevent or treat autoimmune disease comprising:
a) contacting a DAP10 expressing cell with a test compound;
b) assessing the DAP10 expressing cell for modulation of DAP10 activity or expression; and
c) identifying the test compound as a compound that prevents or treats autoimmune disease as one that maintains, enhances or restores DAP10 biological activity in DAP10 expressing cells.
